(19) United States
(12) Patent Application Publication
(10) Pub. No.: US 2009/0074791 A1
(43) Pub. Date: Mar. 19, 2009

(54) Phenylisoquinoline and Phenylquinazoline Derivatives

(76) Inventor: Rene Beerli, Binningen (CH)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 12/202,571

(22) Filed: Sep. 2, 2008

(30) Foreign Application Priority Data
Aug. 30, 2007 (EP) 07115360.5

(51) Int. Cl. A61K 39/395; C07D 403/04; A61K 31/517; A61K 33/06; C07D 401/04; A61K 31/4725

(52) U.S. Cl. 424/158.1; 544/284; 514/266.23; 514/268; 544/249; 546/144; 514/307; 424/722

(57) ABSTRACT

The present invention relates to substituted phenylisoquinoline and phenylquinazoline derivatives and to their pharmaceutical uses in preventing or treating bone conditions which are associated with increased calcium depletion or resorption or in which stimulation of bone formation and calcium fixation in the bone is desirable.

The present invention relates to substituted phenylisoquinoline and phenylquinazoline derivatives and to their pharmaceutical uses in preventing or treating bone conditions which are associated with increased calcium depletion or resorption or in which stimulation of bone formation and calcium fixation in the bone is desirable.

Accordingly the invention provides in one aspect a compound of formula (1) or a pharmaceutically acceptable salt thereof or if possible tautomers thereof:

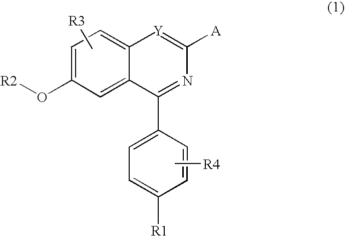

wherein:
Y is CH or N; and
R1 is methyl, ethyl, isopropyl, tert. butyl, or cyclopropyl; and
R2 is methyl, ethyl, propyl, 2-propenyl, or 2-propynyl; and
R3 is hydrogen, chloro, fluoro, bromo, iodo, hydroxy, methoxy, methyl, or trifluoromethyl; and
R4 is hydrogen, chloro, fluoro, bromo, iodo, hydroxy, methoxy, methyl, or trifluoromethyl; and
A is a bi- or tricyclic carbon- or heterocyclic ring system of which one, two, or three cycles can be aryl or heteroaryl and each of which is optionally substituted once or more;
   wherein a bicyclic or tricyclic system as described above contains 7 to 13 ring atoms;
   wherein a bicyclic or tricyclic system as described above contains 7 to 13 ring atoms one or more of which are selected from the group of O, N, S or C═O;
   wherein the optional substituent or substituents being independently selected from the group consisting of phenyl, hydroxyl, trifluoromethyl, methyl, ethyl, branched or unbranched C₃-C₄-alkyl, 2-thiophen, 3-thiophen, hydroxymethyl, hydroxyethyl, methylcarbonyloxymethyl, ethylcarbonyloxyethyl, carboxymethyl, carboxyethyl, methoxycarbonyl, ethoxycarbonyl, branched or unbranched C₃-C₄alkyloxycarbonyl, methoxy, ethoxy, branched or unbranched C₃-C₄-alkyloxy, branched or unbranched C₃-C₅-alkyloxycarbonylmethyl, branched or unbranched C₃-C₅-alkyloxycarbonylethyl, methylcarbonyl, ethylcarbonyl, branched or unbranched C₃-C₄-alkylcarbonyl, phenylcarbonyl, chloro, fluoro, bromo, iodo, cyano, nitro, furanyl, pyrrolyl, thiazolyl, benzothiazolyl, pyridinyl, amino, sulfanyl, sulfonyl, oxycarbonyl, sulfinyl, aminosulfonyl, sulfonylamino, carbonyl, carbonyloxy, carbonyl amino, carboxyl (meaning —COOH), acyl, acylamino, or carbamoyl;
   wherein the optional substituents or substituents as defined above can be again optionally substituted by one or several substituents of the group consisting of methyl, ethyl, branched or unbranched C₃-C₄ alkyl, fluoro, chloro, bromo, iodo, trifluoromethyl, hydroxy, methoxy, amino, alkylamino, dialkylamino, cyano, carboxyl, methylcarboxyl, ethylcarboxyl, branched or unbranched C₃-C₅alkylcarboxyl, and acetyl, or further from 2-hydroxyethyl and 3-hydroxypropyl.

Accordingly the invention provides in another aspect a compound of formula (1′) or its pharmaceutically acceptable salts thereof wherein:
Y, R1, R2, R3, R4 is as defined in formula (1) above; and A in formula (1′) is A′ in formula (1′);
wherein A′ is a bi- or tricyclic carbon- or heterocyclic ring system of which one, two, or three cycles can be aryl or heteroaryl and each of which is optionally substituted once or more;
   wherein a bicyclic or tricyclic system as described above contains 7 to 13 ring atoms;
   wherein a bicyclic or tricyclic system as described above contains 7 to 13 ring atoms one or more of which are selected from the group of O, N, S or C═O;
   wherein the optional substituent or substituents being independently selected from the group consisting of phenyl, hydroxyl, trifluoromethyl, methyl, ethyl, branched or unbranched C₃-C₄-alkyl, 2-thiophen, 3-thiophen, hydroxymethyl, hydroxyethyl, methylcarbonyloxymethyl, ethylcarbonyloxyethyl, carboxymethyl, carboxyethyl, methoxycarbonyl, ethoxycarbonyl, branched or unbranched C₃-C₄alkyloxycarbonyl, methoxy, ethoxy, branched or unbranched C₃-C₄-alkyloxy, branched or unbranched C₃-C₅-alkyloxycarbonylmethyl, branched or unbranched C₃-C₅-alkyloxycarbonylethyl, methylcarbonyl, ethylcarbonyl, branched or unbranched C₃-C₄-alkylcarbonyl, phenylcarbonyl, chloro, fluoro, bromo, iodo, cyano, nitro, furanyl, pyrrolyl, thiazolyl, benzothiazolyl, pyridinyl, amino, sulfanyl, sulfonyl, oxycarbonyl, sulfinyl, aminosulfonyl, sulfonylamino, carbonyl, carbonyloxy, carbonyl amino, carboxyl, acyl, acylamino, and carbamoyl, or further from ethoxycarbonyl, N-(2-hydroxyethyl)-aminocarbonyl, N-(3-hydroxypropyl)-aminocarbonyl, N-methylaminocarbonyl and N,N-dimethylaminocarbonyl.

In a another aspect, the present invention provides a compound of formula (2) or a pharmaceutically acceptable salt thereof:

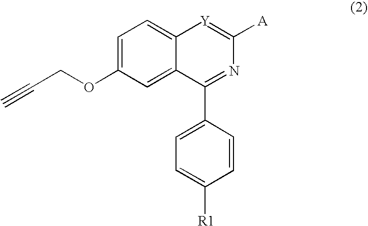

wherein:
R1=R1′ which is isopropyl, tert. butyl, cyclopropyl; and
Y and A is as described in formula (1).

In a further aspect, the present invention provides a compound of formula (3) or a pharmaceutically acceptable salt thereof:

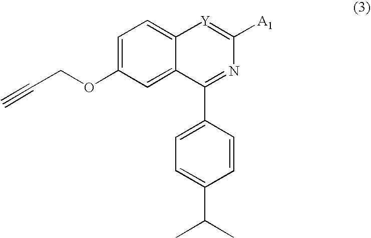

wherein:
Y is CH or N; and
A₁ is being independently selected from the group consisting of

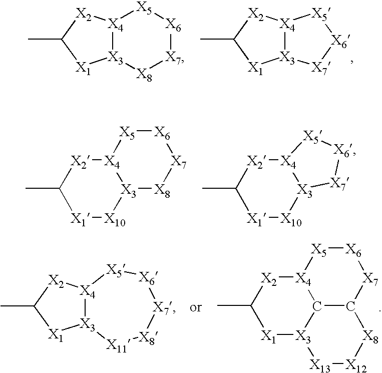

wherein
X₁, X₂ is each independently N, O, S, or C; and
X₁, X₂′ is each independently C, N, or O; and
X₃, X₄ is each independently C or N; and
X₅, X₆, X₇, X₈, X₁₂, X₁₃ is each independently C, N, O, or C═O; and
X₅′, X₆′, X₇′, X₁₁′ is each independently C, N, O, S, C═O; and
X₁₀ is C═O, C, or N; and
wherein the bonds within the cycles are single, double or aromatic; and
wherein the cyclic groups can be optionally substituted as follows:

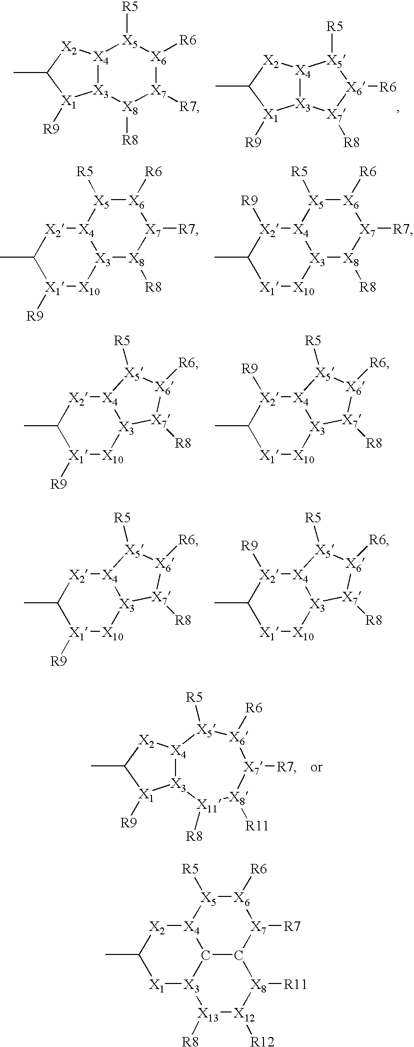

wherein R5, R6, R7, R8, R9, R11, or R12 is each independently selected from the group consisting of hydrogen, phenyl, hydroxyl, trifluoromethyl, methyl, ethyl, branched or unbranched C₃-C₄-alkyl, 2-thiophen, 3-thiophen, hydroxymethyl, hydroxyethyl, methylcarbonyloxymethyl, ethylcarbonyloxyethyl, carboxymethyl, carboxyethyl, methoxy, ethoxy, branched or unbranched C₃-C₄-alkyloxy, branched or unbranched C₃-C₄-alkyloxycarbonylmethyl, branched or unbranched, C₃-C₄-alkyloxycarbonylethyl, methylcarbonyl, ethylcarbonyl, branched or unbranched C₃-C₄-alkylcarbonyl, phenylcarbonyl, chloro, fluoro, bromo, iodo, cyano, nitro, furanyl, thienyl, pyrrolyl, thiazolyl, benzothiazolyl, pyridinyl, amino, sulfanyl, sulfonyl, amino, oxycarbonyl, hydroxyl, sulfinyl, aminosulfonyl, sulfonylamino, carbonyl, carbonyloxy, carbonyl amino, carboxyl, acyl, acylamino, and carbamoyl, or further from ethoxycarbonyl, N-(2-hydroxyethyl)-aminocarbonyl, N-(3-hydroxypropyl)aminocarbonyl, N-methylaminocarbonyl and N,N-dimethylaminocarbonyl; and
wherein neighbored residues of R5, R6, R7, R8, R9, R11, or R12 can form together an alicycle, heterocycle or an aromatic or heteroaromatic cycle which can itself be optionally substituted by methyl, ethyl, branched or unbranched C₃-C₄-alkyl; wherein said aromatic cycle can then form together with the other bicycle e.g. perimidinyl, e.g. 2-perimidinyl or said heteroaromatic cycle can e.g. be phenanthrolinyl, naphtothienyle such as naphto[2,3-b]thienyl or naphtooxazole such as natpho[1,2-d]oxazole.

In a further aspect, the present invention provides a compound of formula (4) or a pharmaceutically acceptable salt thereof:

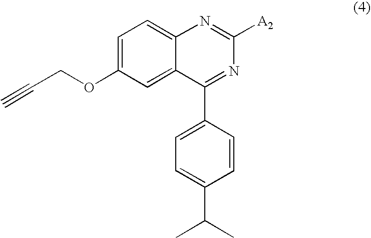

wherein:
A₂ is being independently selected from the group consisting of

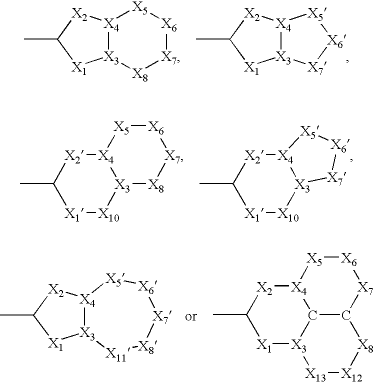

wherein
   X₁, X₂ is each independently N, O, S, or C; and
   X₁′, X₂′ is each independently C, N, or O; and
   X₃, X₄ is each independently C or N; and
   X₅, X₆, X₇, X₈, X₁₂, X₁₃ is each independently C, N, O, or C═O; and
   X₅′, X₆′, X₇′, X₁₁′ is each independently C, N, O, S, C═O; and
   X₁₀ is C═O, C, or N; and
   wherein the bonds within the cycles are single, double or aromatic; and
   wherein the cyclic groups can be optionally substituted as follows:

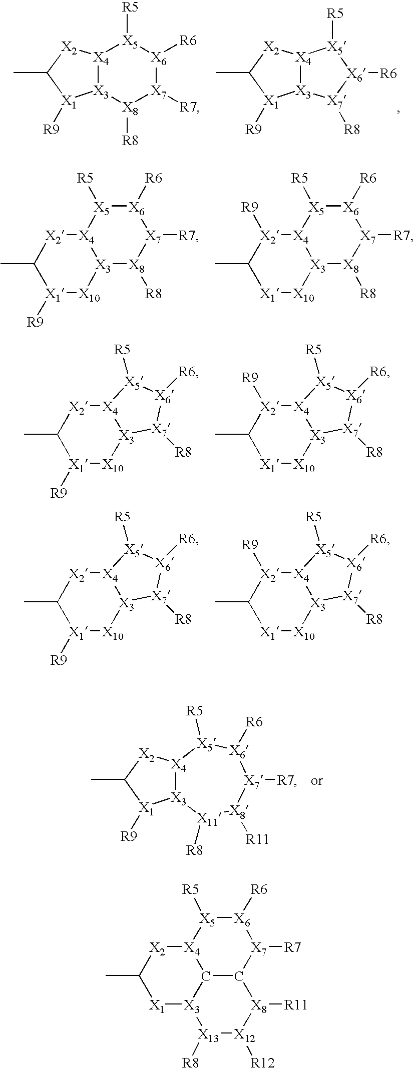

wherein R5, or R8 is hydrogen; and
wherein R6, R7, R11, or R12 is each independently selected from the group consisting of hydrogen, phenyl, hydroxyl, trifluoromethyl, methyl, ethyl, branched or unbranched C₃-C₄-alkyl, 2-thiophen, 3-thiophen, hydroxymethyl, hydroxyethyl, methylcarbonyloxymethyl, ethylcarbonyloxyethyl, carboxymethyl, carboxyethyl, methoxy, ethoxy, branched or unbranched C₃-C₄-alkyloxy, branched or unbranched C₃-C₄-alkyloxycarbonylmethyl, branched or unbranched C₃-C₄-alkyloxycarbonylethyl, methylcarbonyl, ethylcarbonyl, branched or unbranched C₃-C₄-alkylcarbonyl, phenylcarbonyl, chloro, fluoro, bromo, iodo, cyano, and nitro; and
R9 is each independently selected from the group consisting of methyl, ethyl, branched or unbranched C₃-C₄-alkyl; and
wherein neighbored residues of, R6, R7, R9, R11, or R12 can form together an alicycle, heterocycle or an aromatic or heteroaromatic cycle which can itself be optionally substituted by methyl, ethyl, branched or unbranched C₃-C₄-alkyl; wherein said aromatic cycle can then form together with the other bicycle e.g. perimidinyl, e.g. 2-perimidinyl or said heteroaromatic cycle can e.g. be phenanthrolinyl, naphtothienyle such as naphto[2,3-b]thienyl or naphtooxazole such as, natpho[1,2-d]oxazole.

In a further aspect, the present invention provides a compound of formula (5) or a pharmaceutically acceptable salt thereof:

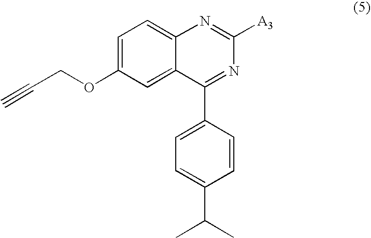

wherein:
A₃ is being independently selected from the group consisting of 5-butyryl-1,6-dimethyl-1H-benzoimidazol-2-yl, 4-methyl-1H-benzoimidazol-2-yl, 6-cyano-1H-benzoimidazol-2-yl, 1-isobutyl-1H-benzoimidazol-2-yl, 5-fluoro-1-methyl-1H-benzoimidazol-2-yl, 5-methoxy-1-methyl-1H-benzoimidazol-2-yl, 4,6-dimethyl-1H-benzoimidazol-2-yl, 1-methyl-1H-benzoimidazol-2-yl, 1-methyl-1H-imidazo[4,5-b]pyridin-2-yl, 1H-imidazo[4,5-b]pyridin-2-yl, 9H-purin-8-yl, 1-phenyl-1H-benzoimidazol-2-yl, 5-butyryl-1-butyl-6-methyl-1H-benzoimidazol-2-yl, 6-trifluoromethyl-1H-benzoimidazol-2-yl, 5-methoxy-1H-benzoimidazol-2-yl, 5-chloro-1H-benzoimidazol-2-yl, 6-ethoxycarbonyl-1H-benzoimidazol-2-yl, 6-benzoyl-1H-benzoimidazol-2-yl, 5-bromo-1H-benzoimidazol-2-yl, 5-fluoro-1H-benzoimidazol-2-yl, 6-chloro-5-fluoro-1H-benzoimidazol-2-yl, 1-methyl-5-trifluoromethyl-1H-benzoimidazol-2-yl, 6-ethoxycarbonyl-1-methyl-1H-benzoimidazol-2-yl, 6-acetyl-1-methyl-1H-benzoimidazol-2-yl, 5,6-difluoro-1H-benzoimidazol-2-yl, 7-acetyl-1-methyl-1H-benzoimidazol-2-yl, 1H-perimidin-2-yl, 4-oxo-3,4-dihydro-quinazolin-2-yl, 5-trifluoromethyl-benzothiazol-2-yl, benzothiazol-2-yl, 5-chloro-benzooxazol-2-yl, and naphtho[1,2-d]oxazol-2-yl; or further from 5-ethoxycarbonyl 3-methyl-3H-benzoimidazole-2-yl, 5-carboxy-3-methyl-3H-benzoimidazole-2-yl, 5-[N-(2-hydroxyethyl)aminocarbonyl]-3-methyl-3H-benzoimidazol-2-yl, 5-(N,N-dimethyl-carbamoyl)-3-methyl-3H-benzimidazol-2-yl and 5-(N-methyl-carbamoyl)-3-methyl-3H-benzimidazol-2-yl.

In a further aspect, the present invention provides a compound selected from the group consisting of:
1-(2-[4-(4-Isopropylphenyl)-6-prop-2-ynyloxy-quinazolin-2-yl]-1,6-dimethyl-1H-benzoimidazol-5-yl)butan-1-one;
4-(4-Isopropyl-phenyl)-2-(4-methyl-1H-benzoimidazol-2-yl)-6-prop2-ynyloxy-quinazoline;
2-[4-(4-isopropyl-phenyl)-6-prop-2-ynyloxy-quinazolin-2-yl]-3H-benzoimidazole-5-carbonitrile;
2-(1-Isobutyl-1H-benzoimidazol-2-yl)-4-(4-isopropyl-phenyl)-6-prop-2-ynyloxy-quinazoline;
2-(5-Fluoro-1-methyl-1H-benzoimidazol-2-yl)-4-(4-isopropyl-phenyl)-6-prop-2-ynyloxy-quinazoline;
4-(4-Isopropyl-phenyl)-2-(5-methoxy-1-methyl-1H-benzoimidazol-2-yl)-6-prop-2-ynyloxy-quinazoline;
2-(4,6-Dimethyl-1H-benzoimidazol-2-yl)-4-(4-isopropyl-phenyl)-6-prop-2-ynyloxy-quinazoline;
4-(4-Isopropyl-phenyl)-2-(1-methyl-1H-benzoimidazol-2-yl)-6-prop-2-ynyloxy-quinazoline;
4-(4-Isopropylphenyl)-2-(1-methyl-1H-imidazo[4,5-b]pyridin-2-yl)-6-prop-2-ynyloxy-quinazoline;
2-(1H-Imidazo[4,5-b]pyridin-2-yl)-4-(4-isopropyl-phenyl)-6-prop-2-ynyloxy-quinazoline;
4-(4-Isopropyl-phenyl)-6-prop-2-ynyloxy-2-(9H-purin-8-yl)-quinazoline;
4-(4-Isopropylphenyl)-2-(1-phenyl-1H-benzoimidazol-2-yl)-6-prop-2-ynyloxy-quinazoline;
1-{1-Butyl-2-[4-(4-isopropyl-phenyl)-6-prop-2-ynyloxy-quinazolin-2-yl]-6-methyl-1H-benzoimidazol-5-yl}-butan-1-one;
4-(4-Isopropyl-phenyl)-6-prop-2-ynyloxy-2-(6-trifluoromethyl-1H-benzoimidazol-2-yl)quinazoline;
4-(4-Isopropyl-phenyl)-2-(6-methoxy-1H-benzoimidazol-2-yl)-6-prop-2-ynyloxy-quinazoline;
2-(6-Chloro-1H-benzoimidazol-2-yl)-4-(4-isopropyl-phenyl)-6-prop-2-ynyloxy-quinazoline;
2-[4-(4-Isopropyl-phenyl)-6-prop-2-ynyloxy-quinazolin-2-yl]-3H-benzoimidazole-5-carboxylic acid ethyl ester;
{2-[4-(4-isopropyl-phenyl)-6-prop-2-ynyloxy-2-yl]-3H-benzoimidazol-5-yl}-phenyl-methanone;
2-(6-Bromo-1H-benzoimidazol-2-yl)-4-(4-isopropyl-phenyl)-6-prop-2-ynyloxy-quinazoline;
2-(6-Fluoro-1H-benzoimidazol-2-yl)-4-(4-isopropyl-phenyl)-6-prop-2-ynyloxy-quinazoline;
2-(6-Chloro-5-fluoro-1H-benzoimidazol-2-yl)-4-(4-isopropyl-phenyl)-prop-2-ynyloxy-quinazoline;
4-(4-Isopropyl-phenyl)-2-(1-methyl-5-trifluoromethyl-1H-benzoimidazol-2-yl)-6-prop-2-ynyloxy-quinazoline;
2-[4-(4-Isopropyl-phenyl)-6-prop-2-ynyloxy-quinazolin-2-yl]-1-methyl-1H-benzoimidazole-carboxylic acid ethyl ester;
1-{2-[4-(4-Isopropyl-phenyl)-6-prop-2-ynyloxy-quinazolin-2-yl]-1-methyl-1H-benzoimidazol-5-yl}-ethanone;
2-(5,6-Difluoro-1H-benzoimidazol-2-yl)-4-(4-isopropyl-phenyl)-6-prop-2-ynyloxy-quinazoline;
1-{2-[4-(4-Isopropylphenyl)-6-prop-2-ynyloxy-quinazolin-2-yl]-3H-benzoimidazol-4-yl}ethanone;
2-[4-(4-Isopropyl-phenyl)-6-prop-2-ynyloxy-quinazolin-2-yl]-1H-perimidine;
4′-(4-isopropyl-phenyl)-6′-prop-2-ynyloxy-3H-[2,2′]biquinazolinyl-4-one;
4-(4-Isopropyl-phenyl)-6-prop-2-ynyloxy-2-(5-trifluoromethyl-benzothiazol-2-yl)quinazoline;
2-Benzothiazol-2-yl-4-(4-isopropyl-phenyl)-6-prop-2-ynyloxy-quinazoline;
2-(6-Chloro-benzooxazol-2-yl)-4-(4-isopropyl-phenyl)-6-prop-2-ynyloxy-quinazoline;
2-[4-(4-Isopropyl-phenyl)-6-prop-2-ynyloxy-quinazolin-2-yl]-naphtho[1,2-d]oxazole;
1-(2-[1-(4-Isopropyl-phenyl)-7-prop-2-ynyloxy-isoquinolin-3-yl]-1,6-dimethyl-1H-benzoimidazol-5-yl)butan-1-one;
1-(4-Isopropyl-phenyl)-7-prop-2-ynyloxy-3-(5-trifluoromethyl-1H-benzoimidazol-2-yl)isoquinoline; or
1-(4-isopropyl-phenyl)-3-(1-methyl-5-trifluoromethyl-1H-benzoimidazol-2-yl)-7-prop-2-ynyloxy-isoquinoline;
   or a pharmaceutically acceptable salt thereof, respectively.

In a further aspect, the present invention provides a compound independently selected from the group consisting of
1-{2-[4-(4-Isopropyl-phenyl)-6-prop-2-ynyloxy-quinazolin-2-yl]-1,6-dimethyl-1H-benzoimidazol-5-yl}-butan-1-one;
4-(4-Isopropyl-phenyl)-2-(4-methyl-1H-benzoimidazol-2-yl)-6-prop2-ynyloxy-quinazoline;
2-[4-(4-Isopropyl-phenyl)-6-prop-2-ynyloxy-quinazolin-2-yl]-3H-benzoimidazole-5-carbonitrile;
2-(1-Isobutyl-1H-benzoimidazol-2-yl)-4-(4-isopropyl-phenyl)-6-prop-2-ynyloxy-quinazoline;
2-(5-Fluoro-1-methyl-1H-benzoimidazol-2-yl)-4-(4 isopropyl-phenyl)-6-prop-2-ynyloxy-quinazoline;
4-(4-Isopropyl-phenyl)-2-(5-methoxy-1-methyl-1H-benzoimidazol-2-yl)-6-prop-2-ynyloxy-quinazoline;
2-(4,6-Dimethyl-1H-benzoimidazol-2-yl)-4-(4-isopropyl-phenyl-6-prop-2-ynyloxy-quinazoline;
4-(4-Isopropyl-phenyl)-2-(1-methyl-1H-benzoimidazol-2-yl)-6-prop-2-ynyloxy-quinazoline;
4-(4-Isopropyl-phenyl)-2-(1-methyl-1H-imidazo[4,5-b]pyridin-2-yl)-6-prop-2-ynyloxy-quinazoline;
2-(1H-Imidazo[4,5-b]pyridin-2-yl)-4-(4-isopropyl-phenyl)-6-prop-2-ynyloxy-quinazoline;
4-(4-Isopropyl-phenyl)-6-prop-2-ynyloxy-2-(9H-purin-8-yl)-quinazoline;
4-(4-Isopropyl-phenyl)-2-(1-phenyl-1H-benzoimidazol-2-yl)-6-prop-2-ynyloxy-quinazoline;
1-{1-Butyl-2-[4-(4-isopropyl-phenyl)-6-prop-2-ynyloxy-quinazolin-2-yl]-6-methyl-1H-benzoimidazol-5-yl}-butan-1-one;
4-(4-Isopropyl-phenyl)-6-prop-2-ynyloxy-2-(6-trifluoromethyl-1H-benzoimidazol-2-yl)quinazoline;
4-(4-Isopropylphenyl)-2-(6-methoxy-1H-benzoimidazol-2-yl)-6-prop-2-ynyloxy-quinazoline;
2-(6-Chloro-1H-benzoimidazol-2-yl)-4-(4-isopropyl-phenyl)-6-prop-2-ynyloxy-quinazoline;
2-[4-(4-Isopropyl-phenyl)-6-prop-2-ynyloxy-quinazolin-2-yl]-3H-benzoimidazole-5-carboxylic acid ethyl ester;
{2-[4-(4-Isopropyl-phenyl)-6-prop-2-ynyloxy-quinazolin-2-yl]-3H-benzoimidazol-5-yl}-phenyl-methanone;
2-(6-Bromo-1H-benzoimidazol-2-yl)-4-(4-isopropyl-phenyl)-6-prop-2-ynyloxy-quinazoline;
2-(6-Fluoro-1H-benzoimidazol-2-yl)-4-(4-isopropyl-phenyl)-4-prop-2-ynyloxy-quinazoline;
2-(6-Chloro-5-fluoro-1H-benzoimidazol-2-yl)-4-(4-isopropyl-phenyl)-6-prop-2-ynyloxy-quinazoline;
4-(4-Isopropyl-phenyl)-2-(1-methyl-5-trifluoromethyl-1H-benzoimidazol-2-yl)-6-prop-2-ynyloxy-quinazoline;
2-[4-(4-Isopropyl-phenyl)-6-prop-2-ynyloxy-quinazolin-2-yl]-1-methyl-1H-benzoimidazole-5-carboxylic acid ethyl ester;
1-{2-[4-(4-Isopropyl-phenyl)-6-prop-2-ynyloxy-quinazolin-2-yl]-1-methyl-1H-benzoimidazol-5-yl}ethanone;
2-(5,6-Difluoro-1H-benzoimidazol-2-yl)-4-(4 isopropyl-phenyl)-6-prop-2-ynyloxy-quinazoline;
1-{-[4-(4-isopropyl-phenyl)-6-prop-2-ynyloxy-quinazolin-2-yl]-3H-benzoimidazol-4-yl}-ethanone;
2-[4-(4-Isopropyl-phenyl)-6-prop-2-ynyloxy-quinazolin-2-yl]-1H-perimidine;
4′-(4-Isopropyl-phenyl)-6′-prop-2-ynyloxy-3H-[2,2′]biquinazolinyl-4-one;
4-(4-Isopropyl-phenyl)-6-prop-2-ynyloxy-2-(5-trifluoromethyl-benzothiazol-2-yl)quinazoline;
2-Benzothiazol-2-yl-4-(4-isopropyl-phenyl)-6-prop-2-ynyloxy-quinazoline;
2-(6-Chloro-benzooxazol-2-yl)-4-(4-isopropyl-phenyl)-6-prop-2-ynyloxy-quinazoline;
   or a pharmaceutically acceptable salt thereof, respectively.

In a further aspect, the present invention provides a compound independently selected from the group consisting of
2-[4-(4-Isopropyl-phenyl)-6-prop-2-ynyloxy-quinazolin-2-yl]-3-methyl-3H-benzoimidazole-5-carboxylic acid ethyl ester,
2-[4-(4-Isopropyl-phenyl)-6-prop-2-ynyloxy-quinazolin-2-yl]-3-methyl-3H-benzoimidazole-carboxylic acid,
2-[4-(4-Isopropyl-phenyl)-6-prop-2-ynyloxy-quinazolin-2-yl]-3-methyl-3H-benzoimidazole-5-carboxylic acid (2-hydroxy-ethyl)-amide,
2-[4-(4-Isopropyl-phenyl)-6-prop-2-ynyloxy-quinazolin-2-yl]-3-methyl-3H-benzoimidazole-5-carboxylic acid dimethylamide; and
2-[4-(4-Isopropyl-phenyl)-6-prop-2-ynyloxy-quinazolin-2-yl]-3-methyl-3H-benzoimidazole-5-carboxylic acid methylamide;
   or a pharmaceutically acceptable salt thereof, respectively.

In a further aspect, the present invention provides a tautomeric form of a compound or pharmaceutically acceptable salts thereof; said compound or salt selected from a compound as defined above.

Pharmaceutically acceptable salts include acid addition salts with conventional acids, for example mineral-acids, e.g. hydrochloric acid, sulfuric or phosphoric acid, or organic acids, for example aliphatic or aromatic carboxylic or sulfonic acids, e.g. acetic, trifluoroacetic, propionic, succinic, glycolic, lactic, malic, tartaric, citric, ascorbic, maleic, fumaric, hydroxylmaleic, pyruvic, pamoic, methanesulfonic, toluenesulfonic, naphthalenesulfonic, sulfanilic or cyclohexylsulfamic acid; also amino acids, such as arginine and lysine. For compounds of the invention having acidic groups, for example a free carboxy group, pharmaceutically acceptable salts also represent metal or ammonium salts, such as alkali metal or alkaline earth metal salts, e.g. sodium, potassium, magnesium or calcium salts, as well as ammonium salts, which are formed with ammonia or suitable organic amines.

Compounds of formula 1, 2, 3, 4, 5 or example compounds (hereinwith “the Agents of the Invention”) which comprise free hydroxyl groups may also exist in the form of pharmaceutically acceptable, physiologically cleavable esters, and as such are included within the scope of the invention. Such pharmaceutically acceptable esters are preferably prodrug ester derivatives, such being convertible by solvolysis or cleavage under physiological conditions to the corresponding agents of the invention which comprise free hydroxyl groups. Suitable pharmaceutically acceptable prodrug esters are those derived from a carboxylic acid, a carbonic acid monoester or a carbamic acid, advantageously esters derived from an optionally substituted lower alkanoic acid or an arylcarboxylic acid.

Acyl is preferably acetyl (methylcarbonyl or, if bound to N, methanecarbonyl), propionyl (ethylcarbonyl or, if bound to N, ethanecarbonyl), butyroyl (n-propylcarbonyl or, if bound to N, n-propane-carbonyl), benzoyl (phenylcarbonyl), methanesulfonyl, ethanesulfonyl, phenylsulfonyl (phenyl-SO₂—) or toluolsulfonyl.

In a further aspect the invention provides a process for preparation of a compound selected from the group consisting of the Agents of the Invention, comprising the step of:

reacting a compound of formula (10)

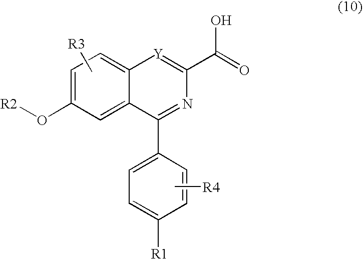

with a compound selected from the group consisting of

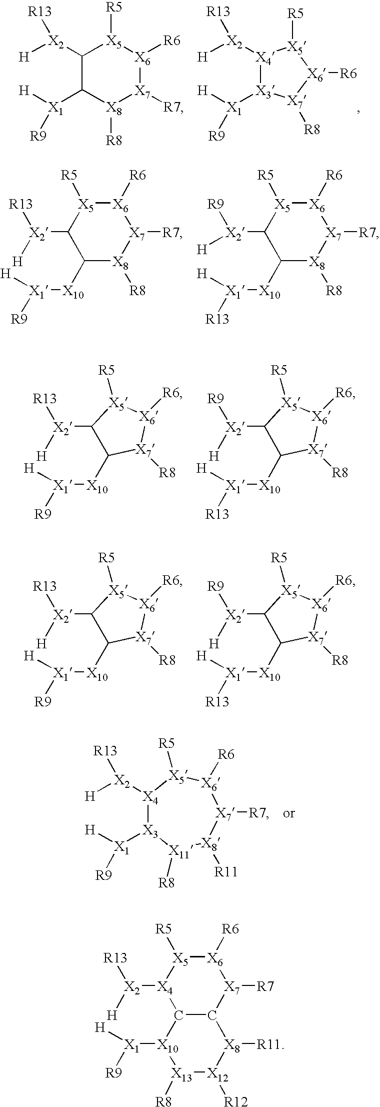

wherein the residues R1 to R12, Y are as defined above; and
wherein the H forms part of an amino, thiol, hydroxy, or basic CHₙ group, the reaction being carried out in the presence of a coupling reagent; and
wherein R13 is an electron lone pair or H, in case R9 is not H,
wherein a compound of formula (10) can e.g. be prepared as described in WO2007/020046.

Alternatively, in cases whereas X₂=N compounds of formula (13), see below, can be prepared by coupling 10 with a suitable precursor containing a nitro group to obtain 11 followed by reduction of the nitro to an amino group to obtain 12 and followed by the cyclization to obtain a compound of formula (13) wherein all the other substituents are defined as described above.

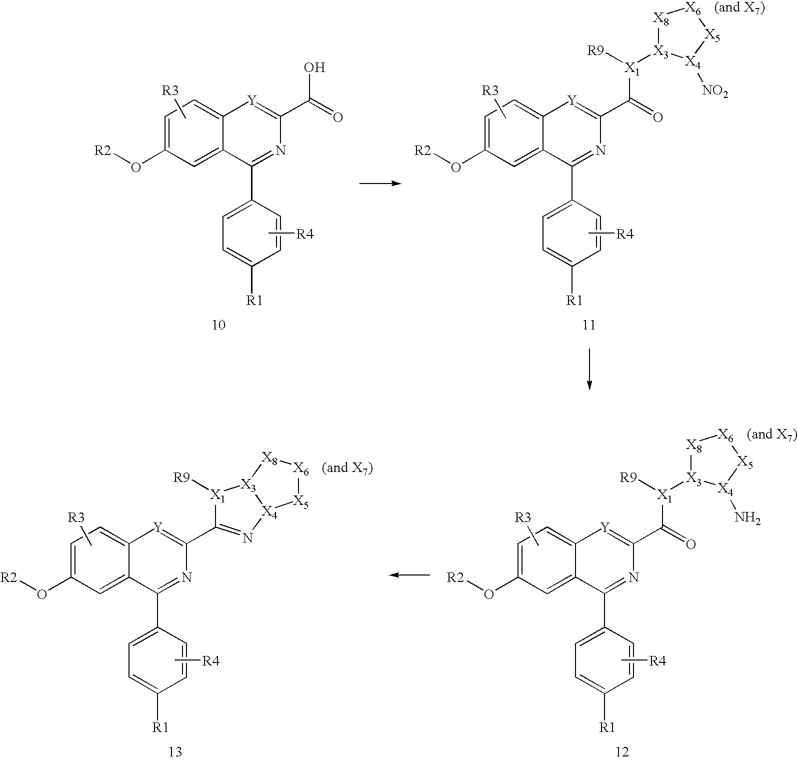

A compound selected from the group consisting of the Agents of the Invention may be converted into salt forms in conventional manner and vice-versa.

A compound selected from the group consisting of the Agents of the Invention can be recovered from the reaction mixture and purified in conventional manner. Isomers, such as enantiomers, may be obtained in conventional manner, e.g. by fractional crystallization or asymmetric synthesis from corresponding asymmetrically substituted, e.g. optically active starting materials.

The synthesis of a compound of the invention, or a pharmaceutically acceptable salt thereof, is also possible in analogy to or by methods described in the examples or above and below in the general description.

The Agents of the Invention, as defined above, e.g., of formula (1) to formula (5), particularly as exemplified, in free or pharmaceutically acceptable acid addition salt form, exhibit pharmacological activity and are useful as pharmaceuticals, e.g. for therapy, in the treatment of diseases and conditions as hereinafter set forth.

It is now well established that controlled treatment of patients with parathyroid hormone (PTH) and analogues and fragments thereof can have a pronounced anabolic effect on bone formation. Thus compounds which promote PTH release, such as the Agents of the Invention may be used for preventing or treating conditions of bone which are associated with increased calcium depletion or resorption or in which stimulation of bone formation and calcium fixation in the bone is desirable.

A suitable and well validated assay to measure the anabolic effect on bone formation is the assay to detect intracellular free calcium, i.e. the method to determine antagonism at the parathyroid Calcium receptor (PCaR) consists in measuring the inhibition of intracellular calcium transients stimulated by extracellular calcium:

CCL39 fibroblasts stably transfected with human PCaR are seeded at 40'000 cells/well into 96-well Viewplates and incubated for 24 hours. Medium is then removed and replaced with fresh medium containing 2 μM Fluo-3 AM (Molecular Probes, Leiden, The Netherlands), In routine experiments, cells are incubated at 37° C., 5% CO₂ for 1 h. Afterwards, plates are washed twice with mHBS and wells are refilled with 100 μl mHBS containing the test compounds. Incubation is continued at room temperature for 15 minutes. To record changes of intracellular free calcium, plates are transferred to fluorescence-imaging plate reader (Molecular Devices, Sunnyvale, Calif., USA). A baseline consisting in 5 measurements of 0.4 seconds each (laser excitation 488 nm) is recorded. Cells are then stimulated with calcium (2.5 mM final), and fluorescence changes recorded over a period of 3 minutes.

When measured in the above assays, Agents of the Invention typically have IC₅₀s in the range from about 5000 nM to about 0.1 nM, preferably from 1000 nM down to about 1 nM or less (e.g. down to 0.1 nM), more preferred compounds, e.g. the compounds as exemplified in the Example Section with Y=N, have all an IC₅₀ which is equal or below 200, e.g. below 100 nM, most preferred have an IC₅₀ of below 10 nM, very most preferred below 2 nM. Such very most preferred compounds are e.g. the following:
1-{2-[4-(4-Isopropyl-phenyl)-6-prop-2-ynyloxy-quinazolin-2-yl]-1,6-dimethyl-1H-benzoimidazol-5-yl}-butan-1-one; 0.26 nM
2-[4-(4-isopropyl-phenyl)-6-prop-2-ynyloxy-quinazolin-2-yl]-3H-benzoimidazole-5-carbonitrile; 0.93 nM
2-(5-Fluoro-1-methyl-1H-benzoimidazol-2-yl)-4-(4-isopropyl-phenyl)-6-prop-2-ynyloxy-quinazoline; 1.1 nM
4-(4-Isopropyl-phenyl)-2-(5-methoxy-1-methyl-1H-benzoimidazol-2-yl)-6-prop-2-ynyloxy-quinazoline; 0.7 nM
4-(4-Isopropyl-phenyl)-2-(1-methyl-1H-benzoimidazol-2-yl)-6-prop-2-ynyloxy-quinazoline; 1.5 nM
4-(4-Isopropyl-phenyl)-6-prop-2-ynyloxy-2-(6-trifluoromethyl-1H-benzoimidazol-2-yl)quinazoline; 0.95 nM
4-(4-Isopropyl-phenyl)-2-(6-methoxy-1H-benzoimidazol-2-yl)-6-prop-2-ynyloxy-quinazoline; 0.96 nM
2-(6-Chloro-1H-benzoimidazol-2-yl)-4-(4-isopropyl-phenyl)-6-prop-2-ynyloxy-quinazoline; 0.53 nM
2-[4-(4-Isopropyl-phenyl)-6-prop-2-ynyloxy-quinazolin-2-yl]-3H-benzoimidazole-5-carboxylic acid ethyl ester; 0.74 nM
{2-[4-(4-Isopropyl-phenyl)-6-prop-2-ynyloxy-quinazolin-2-yl]-3H-benzoimidazol-5-yl}-phenyl-methanone; 0.68 nM
2-(6-Bromo-1H-benzoimidazol-2-yl)-4-(4-isopropyl-phenyl)-6-prop-2-ynyloxy-quinazoline; 0.88 nM
2-(6-Fluoro-1H-benzoimidazol-2-yl)-4-(4-isopropyl-phenyl)-6-prop-2-ynyloxy-quinazoline; 1.6 nM
2-(6-Chloro-5-fluoro-1H-benzoimidazol-2-yl)-4-(4-isopropyl-phenyl)-6-prop-2-ynyloxy-quinazoline; 1.4 nM
4-(4-Isopropyl-phenyl)-2-(1-methyl-5-trifluoromethyl-1H-benzoimidazol-2-yl)-6-prop-2-ynyloxy-quinazoline; 0.63 nM
2-[4-(4-Isopropyl-phenyl)-6-prop-2-ynyloxy-quinazolin-2-yl]-1-methyl-1H-benzoimidazole-5-carboxylic acid ethyl ester; 0.4 nM
1-{2-[4-(4-Isopropyl-phenyl)-6-prop-2-ynyloxy-quinazolin-2-yl]-1-methyl-1H-benzoimidazol-5-yl}-ethanone; 1.0 nM
2-(5,6-Difluoro-1H-benzoimidazol-2-yl)-4-(4-isopropyl-phenyl)-6-prop-2-ynyloxy-quinazoline; 0.44 nM
1-{2-[4-(4-Isopropyl-phenyl)-6-prop-2-ynyloxy-quinazolin-2-yl]-3H-benzoimidazol-4-yl}ethanone. 0.59 nM or
2-[4-(4-isopropyl-phenyl)-6-prop-2-ynyloxy-quinazolin-2-yl]-3-methyl-3H-benzoimidazole-5-carboxylic acid ethyl ester 0.65 nM

Thus in a further aspect the invention includes a method for preventing or treating bone conditions which are associated with increased calcium depletion or resorption or in which stimulation of bone formation and calcium fixation in the bone is desirable in which an effective amount (“effective” meaning especially effective in the treatment of said conditions) of a compound selected from the group consisting of the Agents of the Invention is administered to a patient in need of such treatment.

In a further aspect the invention includes the use of a compound selected from the group consisting of the Agents of the Invention in the manufacture of a medicament for preventing or treating bone conditions which are associated with increased calcium depletion or resorption or in which stimulation of bone formation and calcium fixation in the bone is desirable.

In a further aspect the invention includes the use of a compound selected from the group consisting of the Agents of the Invention in the manufacture of a medicament for treating bone conditions which are associated with increased calcium depletion or resorption or in which stimulation of bone formation and calcium fixation in the bone is desirable.

In a further aspect the invention provides a combination comprising a therapeutically effective amount of a compound selected from the group consisting of the Agents of the Invention and a second drug substance selected from: calcium, a calcitonin or an analogue or derivative thereof, a steroid hormone, a partial estrogen agonist or estrogen-gestagen combination, a SERM (Selective Estrogen Receptor Modulator), vitamin D or an analogue thereof or PTH, a PTH fragment (e.g. PTH 1-34) or a PTH derivative for simultaneous, separate or sequential treatment.

Agents of the Invention are accordingly indicated for preventing or treating all bone conditions which are associated with increased calcium depletion or resorption or in which stimulation of bone formation and calcium fixation in the bone is desirable, e.g. osteoporosis of various genesis (e.g. juvenile, menopausal, post-menopausal, post-traumatic, caused by old age or by corticosteroid therapy or inactivity), fractures, osteopathy, including acute and chronic states associated with skeletal demineralisation, osteo-malacia, periodontal bone loss or bone loss due to arthritis or osteoarthritis or for treating hypoparathyroidism.

Further diseases and disorders which might be prevented or treated include e.g. seizures, stroke, head trauma, spinal cord injury, hypoxia-induced nerve cell damage such as in cardiac arrest or neonatal distress, epilepsy, neurodegenerative diseases such as Alzheimer's disease, Huntington's disease and Parkinson's disease, dementia, muscle tension, depression, anxiety, panic disorder, obsessive-compulsive disorder, post-traumatic stress disorder, schizophrenia, neuroleptic malignant syndrome, congestive heart failure; hypertension; gut motility disorders such as diarrhoea, and spastic colon and dermatological disorders, e.g. in tissue healing, for example burns, ulcerations and wounds.

The Agents of the Invention are particularly indicated for preventing or treating osteoporosis of various genesis.

For all the above uses, an indicated daily dosage is in the range from about 0.03 to about 300 mg, preferably 0.03 to 150; e.g. more preferably from 0.03 to 30, yet more preferably 0.1 to 10 mg, or most preferably from 20 to 150 mg, yet more preferably from 50 to 130 mg of a compound of the invention. Agents of the Invention may, for example, be administered once or twice a day or up to twice a week.

The Agents of the Invention may be administered in free form or in pharmaceutically acceptable salt form. Such salts may be, prepared in conventional manner and exhibit the same order of activity as the free compounds. The present invention also provides a pharmaceutical composition comprising an Agent of the Invention in free base form or in pharmaceutically acceptable salt form in association with a pharmaceutically acceptable diluent or carrier. Such compositions may be formulated in conventional manner. The Agents of the Invention may be administered by any conventional route, for example parenterally e.g. in form of injectable solutions, microemulsions or suspensions, enterally, e.g. orally, for example in the form of tablets or capsules or in a transdermal, nasal or a suppository form.

According to a further embodiment of the invention, the Agents of the Invention may be employed as adjunct or adjuvant to other therapy, e.g. a therapy using a bone resorption inhibitor, for example as in osteoporosis therapy, in particular a therapy employing calcium, a calcitonin or an analogue or derivative thereof, e.g. salmon, eel or human calcitonin, a steroid hormone, e.g. an estrogen, a partial estrogen agonist or estrogen-gestagen combination, a SERM (Selective Estrogen Receptor Modulator) e.g. raloxifene, lasofoxifene, bazedoxifene, arzoxifene, FC1271, Tibolone (Livial®), a RANKL antibody, e.g. denosumab, a cathepsin K inhibitor, vitamin-D or an analogue thereof or PTH, a PTH fragment or a PTH derivative e.g. PTH (1-84), PTH (1-34), PTH (1-36), PTH (1-38), PTH (1-31)NH₂ or PTS 893.

When the Agents of the Invention are administered in conjunction with, e.g. as an adjuvant to bone resorption inhibition therapy, dosages for the co-administered inhibitor will of course vary depending on the type of inhibitor drug employed, e.g. whether it is a steroid or a calcitonin, on the condition to be treated, whether it is a curative or preventive therapy, on the regimen and so forth.

In accordance with the foregoing the present invention further provides:

a) an Agent of the Invention or a pharmaceutically acceptable salt thereof for use as a pharmaceutical;
b) a method for preventing or treating above mentioned disorders and diseases in a subject in need of such treatment, which method comprises administering to said subject an effective amount of an Agent of the Invention or a pharmaceutically acceptable salt thereof;
c) an Agent of the Invention or a pharmaceutically acceptable salt thereof for use in the preparation of a pharmaceutical composition e.g. for use in the method as in b) above.

According to a further embodiment of the invention, the Agents of the Invention may be employed as adjunct or adjuvant to other therapy, e.g. a therapy using a bone resorption inhibitor, for example as in osteoporosis therapy, in particular a therapy employing calcium, a calcitonin or an analogue or derivative thereof, e.g. salmon, eel or human calcitonin, a steroid hormone, e.g. an estrogen, a partial estrogen agonist or estrogen-gestagen combination, a SERM (Selective Estrogen Receptor Modulator) e.g. raloxifene, lasofoxifene, TSE-424, FC1271, Tibolone (Livial®), vitamin D or an analogue thereof or PTH, a PTH fragment or a PTH derivative e.g. PTH (1-84), PTH (1-34), PTH (1-36), PTH (1-38), PTH (1-31)NH₂ or PTS 893.

When the Agents of the Invention are administered in conjunction with, e.g. as an adjuvant to bone resorption inhibition therapy, dosages for the co-administered inhibitor will of course vary depending on the type of inhibitor drug employed, e.g. whether it is a steroid or a calcitonin, on the condition to be treated, whether it is a curative or preventive therapy, on the regimen and so forth.

A highly preferred embodiment of the invention (=falling under any one or more of formulae (1) to (5)) is a compound selected from those mentioned in the subsequent examples, or a pharmaceutically acceptable salt thereof.

The following examples illustrate the invention and do not limit its scope.

### EXAMPLE SECTION

The analytical HPLC conditions are as follows:
Instrument and settings: Agilent 1100 System with G1311A quarternary pump (0.8 ml dead volume), G1313A autosampler (1 μl injection volume), G1316A column compartment (35° C.), G1315A diode array detector (detection by UV absorption at 210 nm-250 nm wave length), G1946A mass spectrometer with APC ionization.
Column: Phenomenex Luna C8, 50×2.0 mm, 3 μm mean particle size or Waters Symmetry C8, 50×2.1 mm, 3.5 μm mean particle size.
Flow rate: 1.0 ml/min.
Linear gradient: 5% B in A to 95% B in A within 2.0 min.
   A: water containing 5% acetonitrile and 0.1% TFA;
   B: acetonitrile containing 0.1% TFA.

### Example 1

### 1-{2-[4-(4-Isopropyl-phenyl)-6-prop-2-ynyloxy-quinazolin-2-yl]-1,6-dimethyl-1H-benzoimidazol-5-yl}-butan-1-one

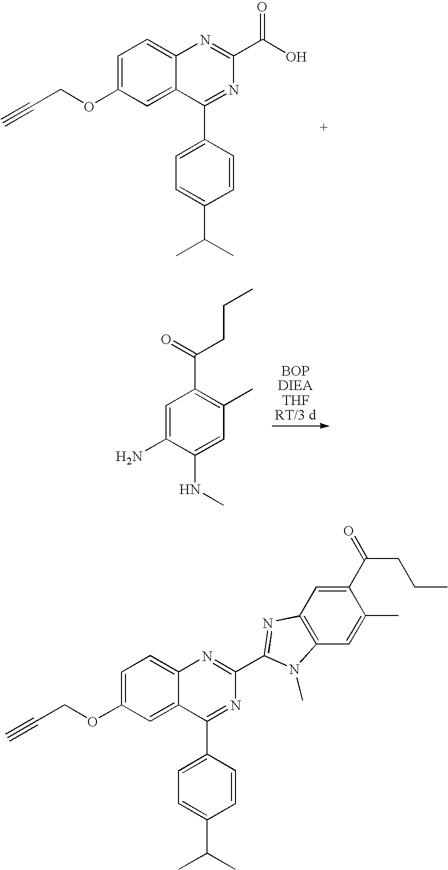

Method A: A mixture of 200 mg (0.58 mmmol) 4-(4-isopropyl-phenyl)-6-prop-2-ynyloxy-quinazoline-2-carboxylic acid [described in WO2007/020046], 120 mg (0.58 mmol) 1-(5-amino-2-methyl-4-methylamino-phenyl)-butan-1-one [described in DE 77-2737462], 380 mg (0.87 mmol) BOP, and 150 μl (0.87 mmol) N-ethyl-diisopropylamine in 2 ml THF is stirred for 3 days at RT. The reaction mixture is purified by reversed phase preparative HPLC using a water/acetonitrile gradient containing 0.1% TFA. The fractions containing the product are extracted with 1 M aqueous NaOH and dichloromethane. After drying over MgSO₄ and evaporation of the organic phases the residue is treated with diethyl ether and, the crystalline product is filtered off to yield the title compound:

¹H NMR (CDCl₃, 400 MHz): 8.37 (s, 1H), 8.25 (d, 1H), 7.90 (d, 2H), 7.67-7.66 (m, 2H), 7.46 (d, 2H), 7.29 (s, 1H), 4.79 (m, 1H), 4.39 (s, 3H), 3.06 (hept, 1H), 2.99 (t, 2H), 2.69 (s, 3H), 2.62 (m, 1H), 1.78 (sext, 2H), 1.36 (d, 6H), 1.01 (t, 3H). HPLC-MS: retention time: 2.30 min (column: Luna), M+1⁺: 517.

### Example 2

### 4-(4-Isopropyl-phenyl)-2-(4-methyl-1H-benzoimidazol-2-yl)-6-prop2-ynyloxy-quinazoline

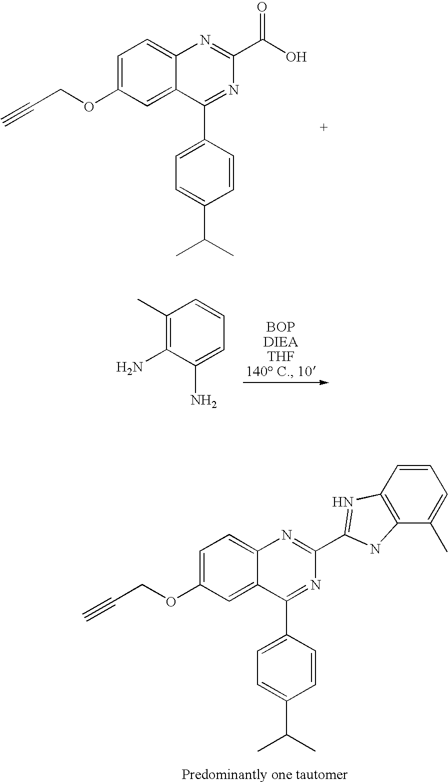

Method B: A mixture of 200 mg (0.58 mmol) 4-(4-isopropyl-phenyl)-6-prop-2-ynyloxy-quinazoline-2-carboxylic acid, 71 mg (0.58 mmol) 2,3-diaminotoluene, 380 mg (0.87 mmol) BOP, and 150 μl (0.87 mmol) N-ethyl-diisopropylamine in 2 ml THF is heated by microwave radiation in a sealed tube for 10 minutes at 140° C. The title compound as free base is obtained after preparative reversed phase HPLC and basic extraction:

¹H NMR (CDCl₃, 400 MHz): 8.24 (d, 1H), 7.85 (d, 2H), 7.64-7.59 (m, 3H), 7.46 (d, 2H), 7.23 (t, 1H), 7.12 (d, 1H), 4.76 (d, 2H), 3.06 (hept, 1H), 2.72 (s, 3H), 2.60 (t, 1H), 1.36 (d, 6H). HPLC-MS: retention time: 2.15 min (column: Luna), M+1⁺: 433.

The following examples are prepared by an analogous procedure:

### Example 3

### 2-[4-(4-Isopropyl-phenyl)-6-prop-2-ynyloxy-quinazolin-2-yl]-3H-benzoimidazole-5-carbonitrile

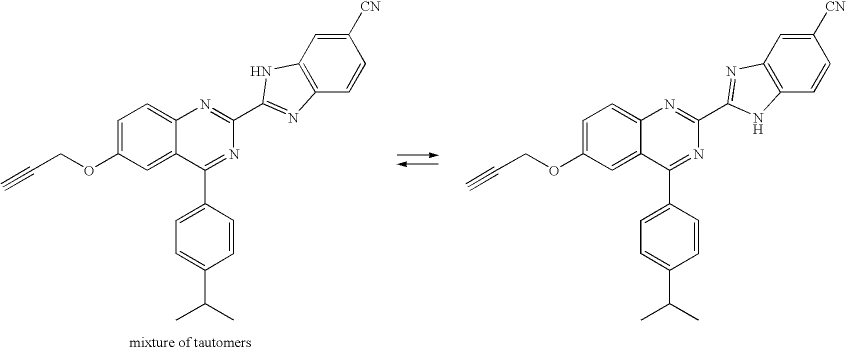

Prepared by method B from 3,4-diaminobenzonitrile.

¹H NMR (DMSO d₆, 600 MHz, 27° C.): 13.65 (br, 1H), 8.33 (br, s, 0.6H), 8.19 (br, d, 1H), 8.05 (br, s, 0.4H), 7.96 (d, 2H), 7.82 (m, 1H), 7.76 (br, d, 0.6H), 7.69-7.67 (m, 2H), 7.64 (br, d, 0.4H), 7.56 (d, 2H), 4.76 (d, 2H), 3.06 (hept, 1H), 2.72 (s, 3H), 2.60 (t, 1H), 1.36 (d, 6H): Mixture of tautomers. ¹H NMR (DMSO d₆, 600 MHz, 121° C.): 13.18 (br, 1H), 8.18-8.10 (m, 2H), 7.90 (d, 2H), 7.85-7.78 (m, 2H), 7.65 (s, 1H), 7.58 (d, 1H), 7.53 (d, 2H), 4.93 (s, 2H), 3.42 (s, 1H), 3.10 (hept, 1H), 1.36 (d, 6H). HPLC-MS: retention time: 2.44 min (column: Luna), 2.40 min (column: Symmetry), M+1⁺: 444.

### Example 4

### 2-(1-Isobutyl-1H-benzoimidazol-2-yl)-4-(4-isopropyl-phenyl)-6-prop-2-ynyloxy-quinazoline

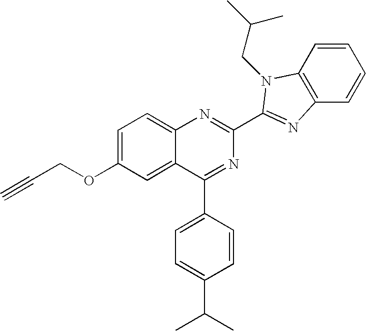

Prepared by method B from N-isobutyl-benzene-1,2-diamine.

¹H NMR (DMSO d₆, 600 MHz, 27° C.): 8.16 (d, 1H), 7.90 (d, 2H), 7.82 (d, 1H), 7.74-7.77 (m, 2H), 7.67 (s, 1H), 7.55 (d, 2H), 7.35 (t, 1H), 7.29 (t, 1H), 4.99 (d, 2H), 4.74 (m, 2H), 3.79 (t, 1H), 3.05 (hept, 1H), 2.15 (m, 1H), 1.30 (d, 6H), 0.79 (d, 6H). HPLC-MS: retention time: 2.53 min (column: Luna), M+1⁺: 475.

### Example 5

### 2-(5-Fluoro-1-methyl-1H-benzoimidazol-2-yl)-4-(4-isopropyl-phenyl)-6-prop-2-ynyloxy-quinazoline

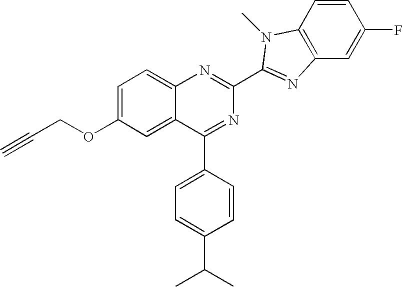

Prepared by method B, but by heating to only 120° C. for 10 minutes, from N-(2-amino fluorophenyl)-N-methylamine.

¹H NMR (DMSO d₆, 400 MHz): 8.17 (d, 1H), 7.90 (d, 2H), 7.79 (dd, 1H), 7.72 (dd, 1H), 7.67 (d, 1H), 7.55 (dd, 1H), 7.53 (d, 2H), 7.24 (td, 1H), 4.98 (d, 2H), 4.26 (s, 3H), 3.77 (t, 1H), 3.03 (hept, 1H), 1.29 (d, 6H). HPLC-MS: retention time: 2.39 min (column: Luna), M+1⁺: 451.

### Example 6

### 4-(4-Isopropyl-phenyl)-2-(5-methoxy-1-methyl-1H-benzoimidazol-2-yl)-6-prop-2-ynyloxy-quinazoline

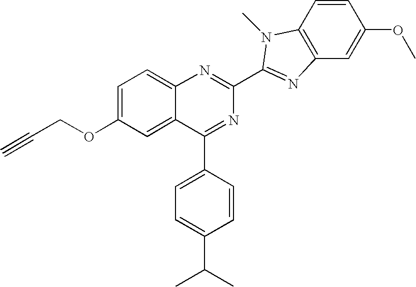

Prepared by method B, but by heating to only 120° C. for 10 minutes, from N-(2-amino methoxyphenyl)-N-methylamine.

¹H NMR (DMSO d₆, 400 MHz): 8.16 (d, 1H), 7.91 (d, 2H), 7.79 (dd, 1H), 7.67 (d, 1H), 7.58 (d, 1H), 7.54 (d, 2H), 7.25 (d, 1H), 7.01 (dd, 1H), 4.98 (d, 2H), 4.23 (s, 3H), 3.81 (s, 3H), 3.77 (t, 1H), 3.04 (hept, 1H), 1.30 (d, 6H). HPLC-MS: retention time: 2.42 min (column: Luna), M+1⁺: 463.

### Example 7

### 2-(4,6-Dimethyl-1H-benzoimidazol-2-yl)-4-(4-isopropyl-phenyl)-6-prop-2-yloxy-quinazoline

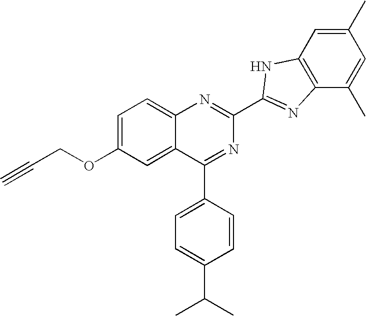

predominately one tautomer

Prepared by method B, but by heating to only 120° C. for 10 minutes, from 3,5-dimethyl benzene-1,2-diamine.

¹H NMR (DMSO d₆, 400 MHz): 12.88 (s, 1H), 8.17 (d, 1H), 7.93 (d, 2H), 7.78 (dd, 1H), 7.62 (d, 1H), 7.54 (d, 2H), 7.19 (s, 1H), 6.86 (s, 1H), 4.96 (d, 2H), 3.76 (t, 1H), 2.57 (s, 3H), 2.39 (s, 3H), 1.31 (s, 6H). HPLC-MS: retention time: 2.44 min (column: Luna), M+1⁺: 447.

### Example 8

### 4-(4-Isopropyl-phenyl)-2-(1-methyl-1H-benzoimidazol-2-yl)-6-prop-2-ynyloxy-quinazoline

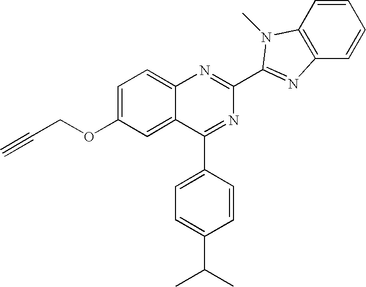

Prepared by method B, but by heating to only 120° C. for 10 minutes, from N-methylbenzene-1,2-diamine.

¹H NMR (DMSO d₆, 400 MHz): 8.18 (d, 1H), 7.91 (d, 2H), 7.80 (dd, 1H), 7.75 (d, 1H), 7.70-7.67 (m, 2H), 7.53 (d, 2H), 7.36 (t, 1H), 7.29 (t, 1H), 4.98 (d, 2H), 4.26 (s, 3H), 3.77 (t, 1H), 3.04 (hept, 1H), 1.30 (d, 6H). HPLC-MS: retention time: 2.38 min-(column: Luna), M+1⁺: 433.

### Example 9

### 4-(4-Isopropyl-phenyl)-2-(1-methyl-1H-imidazo[4,5-b]pyridin-2-yl)-6-prop-2-ynyloxy-quinazoline

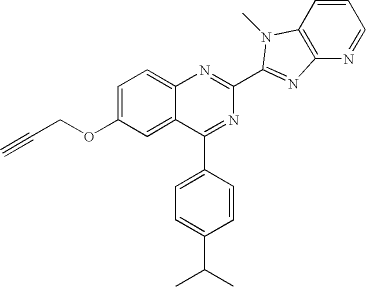

Prepared by method B, but by heating to only 120° C. for 10 minutes, from N(3)-methylpyridine-2,3-diamine [described in Zecchini et al. J. Heterocyclic Chem. 1985, 22, 313].

¹H NMR (DMSO d₆, 400 MHz): 8.52 (dd, 1H), 8.20 (d, 1H), 8.17 (dd, 1H), 7.92 (d, 2H), 7.81 (dd, 1H), 7.69 (d, 1H), 7.53 (d, 2H), 7.38 (dd, 1H), 4.99 (d, 2H), 4.27 (s, 3H), 3.77 (t, 1H), 3.05 (hept, 1H), 1.29 (d, 6H). HPLC-MS: retention time: 2.26 min (column: Luna), M+1⁺: 434.

### Example 10

### 2-(1H-Imidazo[4,5-b]pyridin-2-yl)-4-(4-isopropyl-phenyl)-6-prop-2-ynyloxy-quinazoline

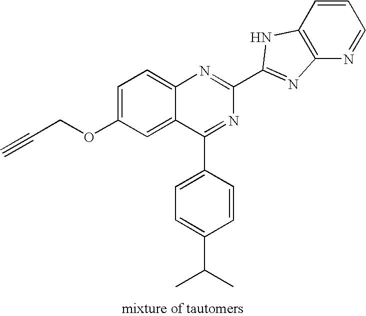

Prepared by method B, but by additional heating to 140° C. for 70 minutes, from pyridine-2,3-diamine.

¹H NMR (DMSO d₆, 400 MHz): 8.45 (d, br, 1H), 8.18 (d, 1H), 8.04 (br, 1H), 7.98 (d, 2H), 7.80 (dd, 1H), 7.68 (d, 1H), 7.54 (d, 2H), 7.31 (dd, 1H), 4.98 (d, 2H), 3.77 (t, 1H), 3.05 (hept, 1H), 1.30 (d, 6H). HPLC-MS: retention time: 2.21 min (column: Luna), M+1⁺: 420.

### Example 11

### 4-(4-Isopropyl-phenyl)-6-prop-2-ynyloxy-2-(9H-purin-8-yl)-quinazoline

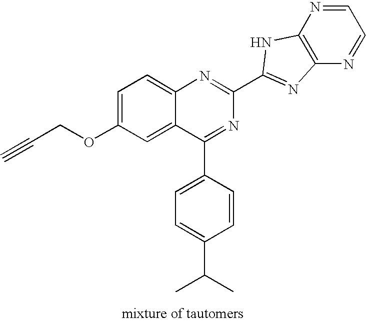

Prepared by method B from pyrimidine-4,5-diamine but heating for 105 minutes at 160° C.

¹H NMR (DMSO d₆, 400 MHz): 14.28 (s, 0.5H), 13.81 (s, 0.5H), 9.23 (s, 0.5H), 9.08 (s, 0.5H), 9.01 (s, 0.5H), 8.95 (s, 0.5H), 8.22-8.17 (m, 1H), 8.01-7.95 (m, 2H), 7.82-7.80 (m, 1H), 7.70 (s, 1H), 7.55-7.53 (m, 2H), 4.99 (s, 2H), 3.77 (s, 1H), 3.05 (hept, 1H), 1.30 (d, 6H). HPLC-MS: retention time: 2.24 min (column: Symmetry), M+1⁺: 421.

### Example 12

### 4-(4-Isopropyl-phenyl)-2-(1-phenyl-1H-benzoimidazol-2-yl)-6-prop-2-ynyloxy-quinazoline

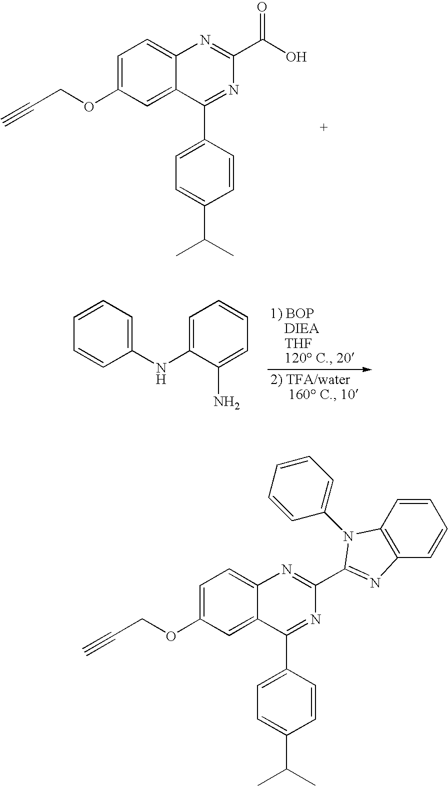

Method C: A mixture of 100 mg (0.29 mmol) 4-(4-isopropyl-phenyl)-6-prop-2-ynyloxy-quinazoline-2-carboxylic acid, 53 mg (0.29 mmol) N-phenyl-1,2-benzenediamine, 190 mg (0.43 mmol) BOP, and 74 μl (0.43 mmol) N-ethyl-diisopropylamine in 1 ml THF is heated for 20 minutes at 120° C. in a sealed tube by microwave radiation. After addition of 1 ml TFA and 1 ml water it is heated for 10 minutes at 160° C. in the microwave oven. The title compound as free base is obtained after preparative reversed phase HPLC and basic extraction.

¹H NMR (CDCl₃, 400 MHz): 8.17 (d, 1H), 8.03 (d, 1H), 7.61-7.59 (m, 2H), 7.56-7.51 (m, 3H), 7.43-7.39 (m, 2H), 7.37 (t, 1H), 7.32 (t, 1H), 7.27-7.24 (m, 5H), 4.76 (d, 2H), 2.98 (hept, 1H), 2.59 (t, 1H), 1.31 (d, 6H). HPLC-MS: retention time: 2.30 min (column: Luna), M+1⁺: 495.

The following examples are prepared by an analogous procedure:

### Example 13

### 1-{1-Butyl-2-[4-(4-isopropyl-phenyl)-6-prop-2-ynyloxy-quinazolin-2-yl]-6-methyl-1H-benzoimidazol-5-yl}-butan-1-one

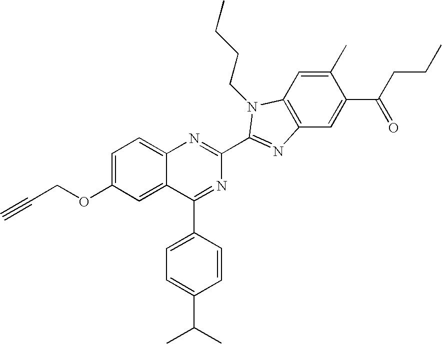

Prepared by method C from 1-(5-amino-4-butylamino-2-methyl-phenyl)butan-1-one which was synthesized in analogy to the preparation of 1-(5-amino-4-methylamino-2-methylphenyl)-butan-1-one [described in DE 77-2737462].

¹H NMR (CDCl₃, 400 MHz): 8.35 (s, 1H), 8.25 (d, 1H), 7.86 (d, 2H), 7.68-7.66 (m, 2H), 7.49 (d, 2H), 7.28 (s, 1H), 4.88 (t, 2H), 4.80 (d, 2H), 3.06 (hept, 1H), 2.98 (t, 2H), 2.70 (s, 3H), 2.62 (t, 1H), 1.94 (quint, 2H), 1.78 (sext, 2H), 1.38 (sext, 2H), 1.36 (d, 6H), 1.01 (t, 3H), 0.89 (t, 3H). HPLC-MS: retention time: 2.45 min (column: Luna), M+1⁺: 559.

### Example 14

### 4-(4-Isopropyl-phenyl)-6-prop-2-ynyloxy-2-(6-trifluoromethyl-1H-benzoimidazol-2-yl)-quinazoline

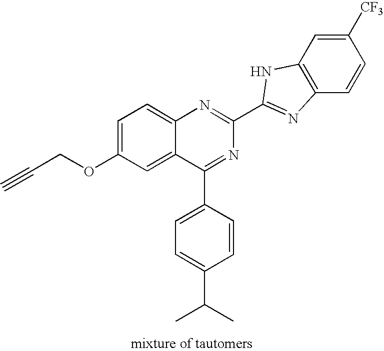

Prepared by method C from 4-(trifluoromethyl)-1,2-phenylenediamine.

¹H NMR (DMSO d₆, 400 MHz, 121° C.): 8.25 (br, 3H), 7.93-7.87 (m, 4H), 7.74 (s, br, 1H), 4.95 (d, 2H), 3.38 (s, br, 1H), 3.08 (hept, 1H), 1.35 (d, 6H). HPLC-MS: retention time: 2.50 min. (column: Luna), M+1⁺: 487

### Example 15

### 4-(4-Isopropyl-phenyl)-2-(6-methoxy-1H-benzoimidazol-2-yl)-6-prop-2-ynyloxy-quinazoline

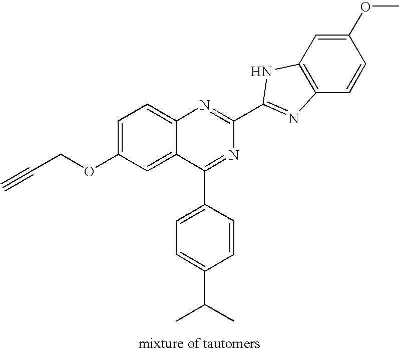

Prepared by method C from 4-methoxy-o-phenylenediamine.

¹H NMR (CDCl₃, 400 MHz): 8.19 (d, 1H), 7.82 (d, 2H), 7.60-7.57 (m, 3H), 7.43 (d, 2H), 7.11 (br, 1H), 6.93 (d, 1H), 4.74 (d, 2H), 3.84 (s, 3H), 3.04 (hept, 1H), 2.59 (t, 1H), 1.34 (d, 6H). HPLC-MS: retention time: 2.14 min (column: Luna), M+1⁺: 449.

### Example 16

### 2-(6-Chloro-1H-benzoimidazol-2-yl)-4-(4-isopropyl-phenyl)-6-prop-2-ynyloxy-quinazoline

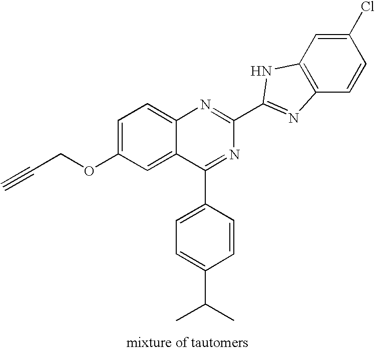

Prepared by method C from 4-chloro-o-phenylenediamine.

¹H NMR (DMSO d₆, 600 MHz): 8.18 (br, 1H), 7.95 (br, 2H), 7.81 (br, 2H), 7.67 (br, 2H), 7.56 (d, 2H), 7.30 (m, 1H), 4.98 (s, 2H), 3.78 (s, 1H), 3.06 (hept, 1H), 1.31 (d, 6H). HPLC-MS: retention time: 2.40 min (column: Luna), 2.34 (column: Symmetry), M+1⁺: 453 (isotope pattern for 1 Cl atom).

### Example 17

### 2-[4-(4-Isopropyl-phenyl)-6-prop-2-ynyloxy-quinazolin-2-yl]-3H-benzoimidazole-5-carboxylic acid ethyl ester

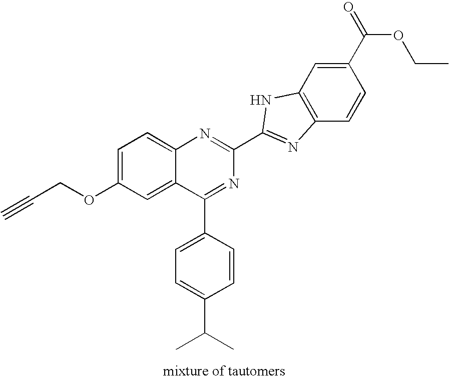

Prepared by method C from ethyl 3,4-diaminobenzoate.

¹H NMR (DMSO d₆, 400 MHz): 13.46 (br, 1H), 8.36 (br, 0.5H), 8.24 (br (0.5H), 8.18 (d, 1H), 7.94 (d, 2H), 7.91-7.79 (m, 3H), 7.69-7.65 (m, 1H), 7.55 (d, 2H), 4.97 (s, 2H), 4.34 (q, 2H), 3.77 (s, 1H), 3.05 (m, 1H), 1.34 (t, 3H), 1.30 (d, 6H). HPLC-MS: retention time: 2.42 min (column: Luna), M+1⁺: 491.

### Example 18

### {2-[4-(4-isopropyl-phenyl)-6-prop-2-ynyloxy-quinazolin-2-yl]-3H-benzoimidazol-5-yl}phenyl-methanone

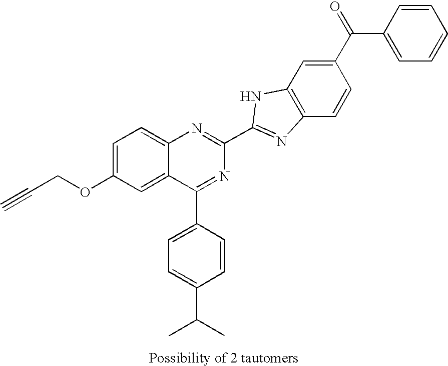

Prepared by method C from 3,4-diaminobenzophenone.

¹H NMR (CDCl₃, 400 MHz): 8.26-8.21 (m, 2H), 7.89-7.82 (m, 6H), 7.67-7.57 (m, 3H), 7.50-7.44 (m, 4H), 4.78 (d, 2H), 3.06 (hept, 1H), 2.62 (t, 1H), 1.36 (d, 6H). HPLC-MS: retention time: 2.46 min (column: Luna), M+1⁺: 523.

### Example 19

### 2-(6-Bromo-1H-benzoimidazol-2-yl)-4-(4-isopropyl-phenyl)-6-prop-2-ynyloxy-quinazoline

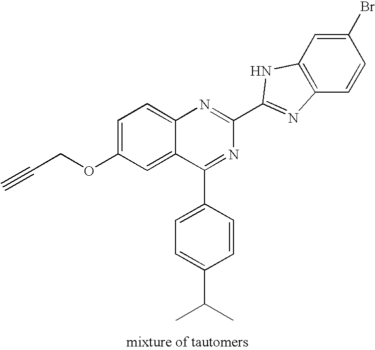

Prepared by method C from 4-bromo-o-phenylenediamine.

¹H NMR (DMSO d₆, 400 MHz): 13.3 (br, 1H), 8.18 (br, 1H), 7.95 (br, 2H), 7.81-7.80 (br, 2H), 7.67 (br, 1H), 7.55 (d, br, 2H), 7.43-7.39 m, br, 2H), 4.98 (br, 2H), 3.78 (br, 1H), 3.06 (hept, 1H), 1.31 (d, 6H). HPLC-MS: retention time: 2.41 min (column: Luna), M+1⁺: 497/499 (isotope pattern for 1 Br atom).

### Example 20

### 2-(6-Fluoro-1H-benzoimidazol-2-yl)-4-(4-isopropyl-phenyl)-6-prop-2-ynyloxy-quinazoline

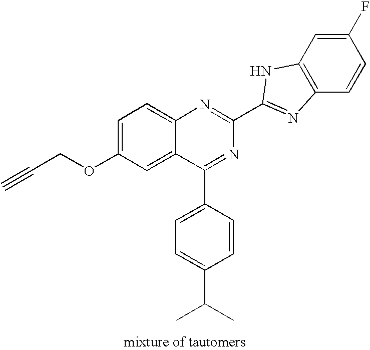

Prepared by method C from 3,4-diaminobenzene.

¹H NMR (DMSO d₆, 400 MHz): 13.23 (br, 0.5H), 13.20 (br, 0.5H), 8.14 (d, 1H), 7.94 (d, 2H), 7.79 (dd, 1H), 7.75 (br, 0.5H), 7.64 (br, 1H), 7.60-7.53 (m, 1H), 7.54 (d, 2H), 7.32 (d, br, 0.5H), 7.15 (t, 0.5H), 7.10 (t, 0.5H), 4.97 (d, 2H), 3.77 (t, 1H), 3.05 hept, 1H), 1.30 (d, 6H). HPLC-MS: retention time: 2.35 min (column: Luna), M+1⁺: 437.

### Example 21

### 2-(6-Chloro-5-fluoro-1H-benzoimidazol-2-yl)-4-(4-isopropyl-phenyl)-6-prop-2-ynyloxy-quinazoline

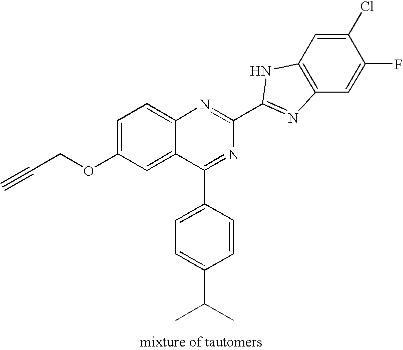

Prepared by method C from 4-chloro-5-fluoro-1,2-phenylenediamine.

¹H NMR (DMSO d₆, 400 MHz): 13.37 (s, 0.5H), 13.32 (s, 0.5H), 8.16 (d, 1H), 7.98 (d, 0.5H), 7.95-7.93 (m, 2.5H), 7.81-7.78 (m, 1.5H), 7.70 (d, 0.5H), 7.65 (d 1H), 7.56-7.51 (m, 3H), 4.97 (d, 2H), 3.76 (t, 1H), 3.05 (hept, 1H), 1.30 (d, 6H). HPLC-MS: retention time: 2.50 min (column: Luna), M+1⁺: 471 (isotope pattern for 1 Cl atom).

### Example 22

### 4-(4-Isopropyl-phenyl)-2-(1-methyl-5-trifluoromethyl-1H-benzoimidazol-2-yl)-6-prop-2-ynyloxy-quinazoline

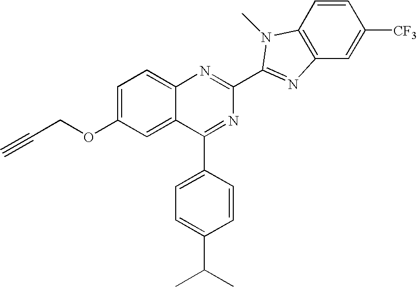

Prepared by method C from N-methyl-4-(trifluoromethyl)benzene-1,2-diamine but heating only to 140° C. instead of 160° C. in the second step.

¹H NMR (DMSO d₆, 400 MHz): 8.20 (d, 1H), 8.14 (s, 1H), 7.94-7.91 (m, 3H), 7.81 (dd, 1H), 7.70-7.67 (m, 2H), 7.54 (d, 2H), 4.99 (d, 2H), 4.31 (s, 3H), 3.78 (t, 1H), 3.04 (hept, 1H), 1.30 (d, 6H). HPLC-MS: retention time: 2.53 min (column: Luna), M+1⁺: 501.

### Example 23

### 2-[4-(4-Isopropyl-phenyl)-6-prop-2-ynyloxy-quinazolin-2-yl]-1-methyl-1H-benzoimidazole-5-carboxylic acid ethyl ester

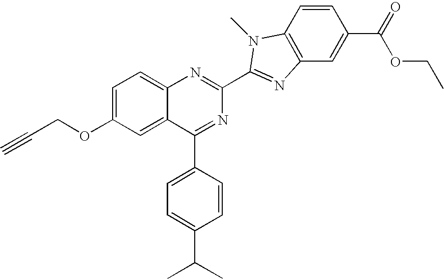

Prepared by method C from 3-amino-4-methylamino-benzoic acid ethyl ester but heating only to 140° C. instead of 160° C. in the second step.

¹H NMR (DMSO d₆, 400 MHz): 8.19 (d, 2H), 7.97 (dd, 1H), 7.91 (d, 2H), 7.82-7.79 (m, 2H), 7.67 (d, 1H), 7.53 (d, 2H), 4.98 (d, 2H), 4.34 (q, 2H), 4.28 (s, 3H), 3.77 (t, 1H), 3.03 (hept, 1H), 1.35 (t, 3H), 1.29 (d, 6H). HPLC-MS: retention time: 2.46 min (column: Luna), M+1⁺: 505.

### Example 24

### 1-{2-[4-(4-Isopropyl-phenyl)-6-prop-2-ynyloxy-quinazolin-2-yl]-1-methyl-1H-benzoimidazol-5-yl}ethanone

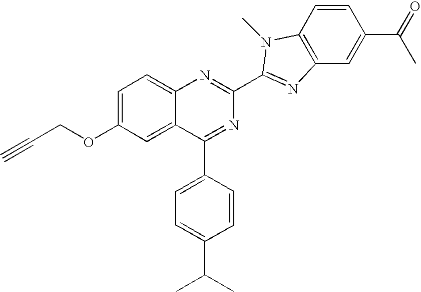

Prepared by method C from 3-amino-4-methylamino-benzoic acid ethyl ester but heating only to 140° C. instead of 160° C. in the second step.

¹H NMR (DMSO d₆, 400 MHz): 8.42 (d, 1H), 8.19 (d, 1H), 7.98 (dd, 1H), 7.91 (d, 2H), 7.80 (dd, 1H), 7.79 (d, 1H), 7.68 (d, 1H), 7.54 (d, 2H), 4.99 (d, 2H), 4.29 (s, 3H), 3.78 (t, 1H), 3.04 (hept, 1H), 2.66 (s, 3H), 1.29 (d, 6H). HPLC-MS: retention time: 2.37 min (column: Luna), M+1⁺: 475.

### Example 25

### 2-(5,6-Difluoro-1H-benzoimidazol-2-yl)-4-(4-isopropyl-phenyl)-6-prop-2-ynyloxy-quinazoline

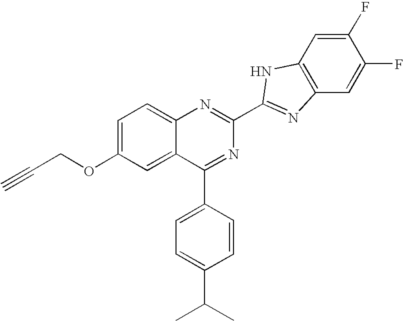

Prepared by method C from 4,5-difluoro-benzene-1,2-diamine but heating only to 140° C. instead of 160° C. in the second step.

¹H NMR (DMSO d₆, 400 MHz): 13.31 (s, 1H), 8.14 (d, 1H), 7.93 (d, 2H), 7.81 (d, 1H), 7.79 (dd, 1H), 7.65 (d, 1H), 7.54 (d, 2H), 7.51 (d, 1H), 4.97 (d, 2H), 3.76 (t, 1H), 3.05 (hept, 1H), 1.30 (d, 6H). HPLC-MS: retention time: 2.36 min (column: Symmetry), M+1⁺: 455.

### Example 26

### 1-{2-[4-(4-Isopropyl-phenyl)-6-prop-2-ynyloxy-quinazolin-2-yl]-3H-benzoimidazol-4-yl}-ethanone

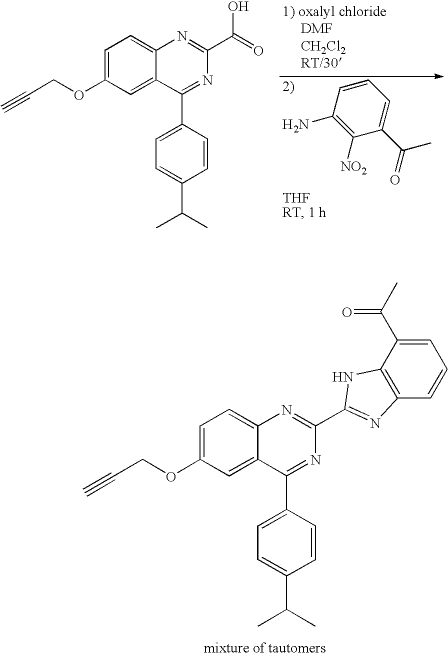

To a solution of 500 mg (1.44 mmol) 4-(4-isopropyl-phenyl)-6-prop-2-ynyloxy-quinazoline-2-carboxylic acid in 8 ml dichloromethane are added 378 μl (4.33 mmol) oxalyl chloride and 24.7 μl (0.32 mmol) DMF. After 30 minutes the solvent is evaporated and the residue is three times dissolved in toluene and evaporated to remove traces of unreacted oxalyl chloride. The residue is dissolved in 8 ml THF and 260 mg (1.44 mmol) 1-(3-amino-2-nitro-phenyl)ethanone [Wm. A. Waters, J. Chem. Soc. 1945, 629] are added. After stirring for 1 h at RT the reaction mixture is poured onto saturated aqueous NaHCO₃ and extracted with dichloromethane. The organic phases are dried over MgSO₄ and evaporated. Diethyl ether is added and the suspension is stirred for 15 minutes. The resulting crystals are filtered off. At 60° C. a suspension of these crystals in 5 ml Ethanol are added to a suspension of 362 mg (6.48 mmol) iron powder in 5 ml ethanol and 179 μl (1.81 mmol) concentrated aqueous HCl. After 30 minutes 440 μl (2.2 mmol) of an aqueous solution (27%) of ammonium chloride are added. After 3 h stirring at 60° C., again 362 mg (6.48 mmol) iron powder and 179 μl (1.81 mmol) concentrated aqueous HCl are added and heating at 60° C. is continued for 3 days. The reaction mixture is diluted with ethyl acetate and filtered through Hyflo. The filtrate is washed with water, dried over MgSO₄ and completely evaporated. The crude product is purified by chromatography on silica applying a hexane/ethyl acetate gradient. All the fractions containing the product are evaporated and diethyl ether is added. After stirring for a while the resulting crystals of the title compound are filtered off.

¹H NMR (DMSO d₆, 600 MHz): 13.52 (br, 0.5H), 12.01 (br, 0.5H), 8.22 (d, 1H), 8.11 (br, 0.5H), 8.08 (br, 0.5H), 7.96 (d, 2H), 7.87 (br, 0.5H), 7.81 (dd, 1H), 7.75 (br, 0.5H), 7.67 (d, 1H), 7.57 (d, 2H), 7.44 (br, 1H), 4.99 (d, 2H), 3.79 (t, 1H), 3.09 (br, 1.5H), 3.06 (hept, 1H), 2.74 (br, 1.5H), 1.32 (d, 6H). HPLC-MS: retention time: 2.40 min (column: Luna), M+1⁺: 461.

### Example 27

### 2-[4-(4-Isopropyl-phenyl)-6-prop-2-ynyloxy-quinazolin-2-yl]-1H-perimidine

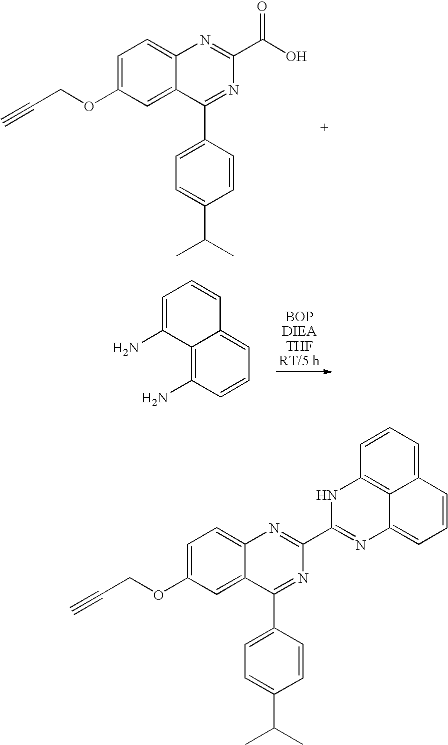

A mixture of 100 mg (0.29 mmol) 4-(4-isopropyl-phenyl)-6-prop-2-ynyloxy-quinazoline-2-carboxylic acid, 46 mg (0.29 mmol) 1,8-diaminonaphthalene, 190 mg (0.43 mmol) BOP, and 74 μl N-ethyl-diisopropylamine is stirred at RT for 5 h. The reaction mixture is purified by reversed phase HPLC. The fractions containing the product are extracted with 1 M aqueous NaOH and dichloromethane. The organic phases are dried over MgSO₄ and evaporated. The residue is taken up in a little amount of ethyl acetate and the crystalline product is filtered off to yield the title compound.

¹H NMR (DMSO d₆, 400 MHz): 8.32 (d, 1H), 7.97 (d, 2H), 7.92 (dd, 1H), 7.74 (d, 1H); 7.58 (d, 2H), 7.35-7.30 (m, 4H), 7.17-7.12 (m, 2H), 5.03 (d, 2H), 3.79 (t, 1H), 3.06 (hept, 1H), 1.31 (d, 6H). HPLC-MS: retention time: 2.23 min (column: Luna), M+1⁺: 469.

### Example 28

### 4′-(4-isopropyl-phenyl)-6′-prop-2-ynyloxy-3H-[2,2′]biquinazolinyl one

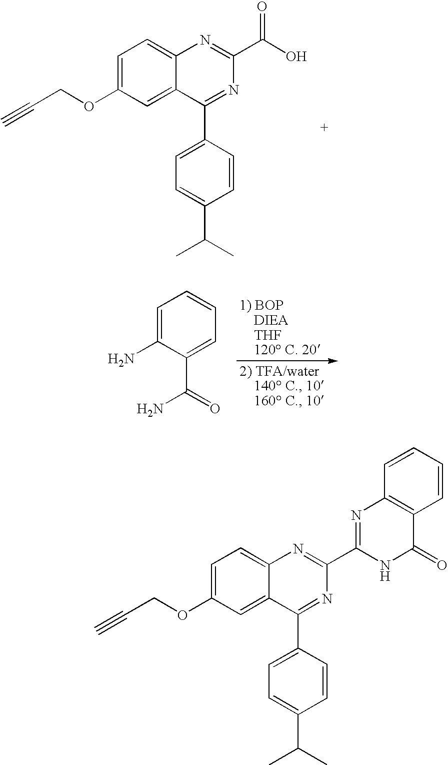

A mixture of 200 mg (0.58 mmol) 4-(4-isopropyl-phenyl)-prop-2-ynyloxy-quinazoline-2-carboxylic acid, 79 mg (0.58 mmol) 2-amino-benzamide, 380 mg (0.87 mmol) BOP, and 150 μl (0.87 mmol) N-ethyldiisopropylamine in 2 ml THF is heated for 20 minutes at 120° C. in a sealed tube by microwave radiation. After addition of 1 ml TFA and 1 ml water the reaction mixture is heated in a microwave oven for 10 minutes at 140° C. followed by 10 minutes at 160° C. The title compound is isolated by chromatography on silica applying a hexane/ethyl acetate gradient.

¹H NMR (DMSO d₆, 400 MHz): 12.21 (s, 1H), 8.24 (d, 1H), 8.21 (d, 1H), 7.98 (d, 2H), 7.89-7.88 (m, 2H), 7.82 (dd, 1H), 7.67 (d, 1H), 7.61 (m, 1H), 7.53 (d, 2H), 4.99 (d, 2H), 3.78 (t, 1H), 3.04 (hept, 1H), 1.29 (d, 6H). HPLC-MS: retention time: 2.58 min (column: Luna), M+1⁺: 447.

### Example 29

### 4-(4-isopropyl-phenyl)-6-prop-2-ynyloxy-2-(5-trifluoromethyl-benzothiazol-2-yl)quinazoline

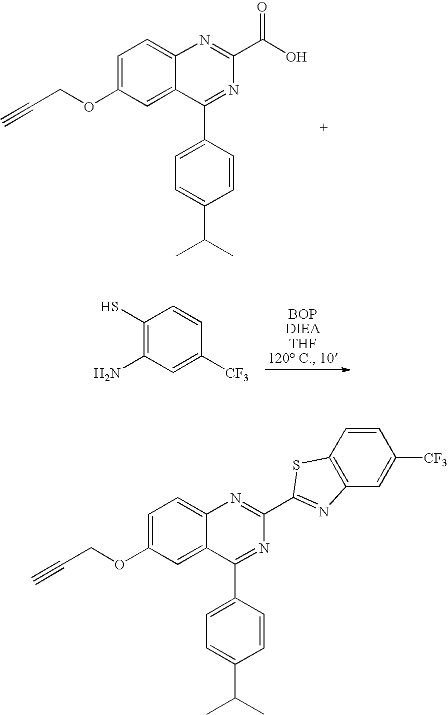

A mixture of 100 mg (0.29 mmol) 4-(4-isopropyl-phenyl)-6-prop-2-ynyloxy-quinazoline-2-carboxylic acid, 66 mg (0.29 mmol) 2-mercapto-5-trifluoromethyl-phenyl-ammonium chloride, 190 mg (0.43 mmol) BOP, and 74 μl (0.43 mmol) N-ethyl-diisopropylamine in 1 ml THF is heated for 10′ at 120° C. in a sealed tube by microwave radiation. The reaction mixture is added to 0.1 M aqueous NaOH solution and extracted with dichloromethane. The organic layers are dried over MgSO₄ and completely evaporated. The product is purified by preparative HPLC. The fractions containing the product are extracted with dichloromethane/1 M NaOH. The organic phases are dried over MgSO₄ and completely evaporated. To the residue, ether is added and the crystalline product is filtered off to yield the title compound.

¹H NMR (DMSO d₆, 400 MHz): 8.54 (s, 1H), 8.44 (d, 1H), 8.21 (d, 1H), 7.91 (d, 2H), 7.84 (dd, 1H), 7.81 (dd, 1H), 7.67 (d, 1H), 7.55 (d, 2H), 4.98 (d, 2H), 3.77 (t, 1H), 3.04 (hept, 1H), 1.30 (d, 6H). HPLC-MS: retention time: 3.02 min (column: Luna), M+1⁺: 504.

### Example 30

### 2-Benzothiazol-2-yl-4-(4-isopropyl-phenyl)-6-prop-2-ynyloxy-quinazoline

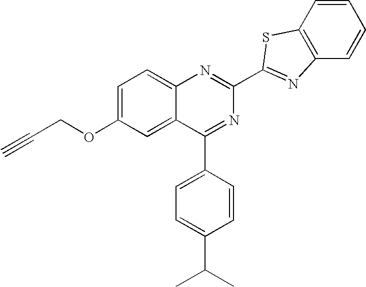

Prepared from 2-aminothiophenol analogously to the example immediately above but additional heating at 140° C. for 70 minutes is necessary.

¹H NMR (DMSO d₆, 400 MHz): 8.20-8.17 (m, 3H), 7.91 (d, 2H), 7.80 (dd, 1H), 7.67 (d, 1H), 7.61-7.51 (m, 4H), 4.98 (d, 2H), 3.77 (t, 1H), 3.05 (hept, 1H), 1.30 (d, 6H). HPLC-MS: retention time: 2.87 min (column: Luna), M+1⁺: 436.

### Example 31

### 2-(6-Chloro-benzooxazol-2-yl)-4-(4-isopropyl-phenyl)-6-prop-2-ynyloxy-quinazoline

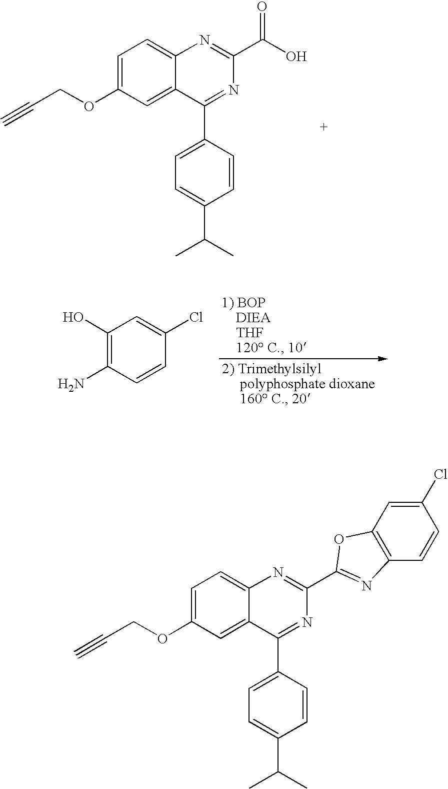

A mixture of 100 mg (0.29 mmol) 4-(4-isopropyl-phenyl-6-prop-2-ynyloxy-quinazoline-2-carboxylic acid, 41 mg (0.29 mmol) 2-amino-5-chlorophenol, 190 mg (0.43 mmol) BOP, and 74 μl (0.43 mmol) N-ethyl-diisopropylamine in 1 ml THF is heated for 10′ at 120° C. in a sealed tube by microwave radiation. The reaction mixture is added to 0.1 M aqueous NaOH solution and extracted with dichloromethane. The organic layers are dried over MgSO₄ and completely evaporated. The crude intermediate is taken up into 2 ml dioxane and 500 μl trimethylsilyl polyphosphate are added. The reaction mixture is heated at 160° C. for 20′ in a sealed tube by microwave radiation. Extraction with 0.1 M aqueous NaOH/dichloromethane followed by purification by preparative HPLC affords the title compound.

¹H NMR (DMSO d₆, 400 MHz): 8.22 (d, 1H), 8.14 (d, 1H), 7.93 (d, 1H), 7.89 (d, 2H), 7.82 (dd, 1H), 7.67 (d, 1H), 7.55-7.51 (m, 3H), 4.99 (d, 2H), 3.77 (t, 1H), 3.04 (hept, 1H), 1.29 (d, 6H). HPLC-MS: retention time: 2.90 min (column: Luna), M+1⁺: 454 (isotope pattern for 1 Cl atom).

The following example is prepared by, an analogous procedure:

### Example 32

### 2-[4-(4-Isopropyl-phenyl)-6-prop-2-ynyloxy-quinazolin-2-yl]-naphtho[1,2-d]oxazole

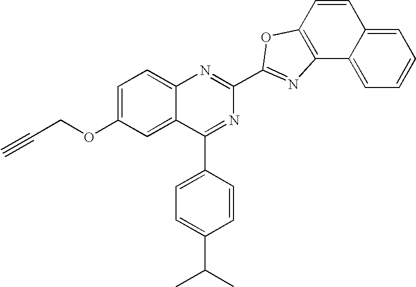

Prepared from 1-amino-2naphthol hydrochloride.

¹H NMR (DMSO d₆, 400 MHz): 8.52 (d, 1H), 8.25 (d, 1H), 8.14 (d, 1H), 8.07 (s, 2H), 7.90 (d, 2H), 7.82 (dd, 1H), 7.76 (t, 1H), 7.65-7.62 (m, 2H), 7.55 (d, 2H), 4.98 (d, 2H), 3.76 (t, 1H), 3.05 hept, 1H), 1.31 (d, 6H). HPLC-MS: retention time: 2.90 min (column: Luna), M+1⁺: 470.

### Example 33

### 1-{2-[1-(4-Isopropyl-phenyl)-7-prop-2-ynyloxy-isoquinolin-3-yl]-1,6-dimethyl-1H-benzoimidazol-5-yl}butan-1-one

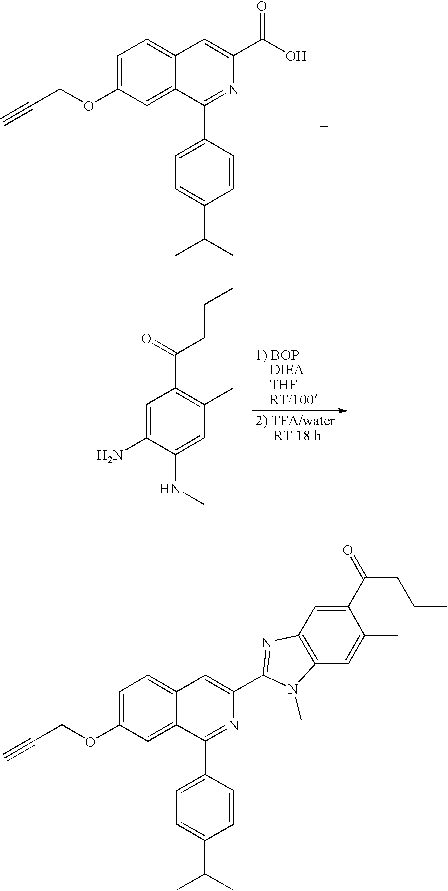

A mixture of 72 mg (0.21 mmol) 1-(4 isopropyl-phenyl)-7-prop-2-ynyloxy-isoquinoline-3-carboxylic acid [described in WO2007/020046], 43 mg (0.21 mmol) 1-(5-amino-2-methyl-4-methylamino-phenyl)-butan-1-one, 54 μl (0.31 mmol) N-ethyl-diisopropylamine, and 138 mg (0.31 mmol) BOP in 1 ml THF is stirred for 100 minutes at RT before 1 ml water and 1 ml TFA are added and stirring is continued for 18 h. The reaction mixture is diluted with dichloromethane and extracted with 1 M aqueous NaOH. The organic layers are dried over MgSO₄ and completely evaporated. The residue is chromatographed on silica applying a hexane/ethyl acetate gradient. The fractions containing the product are evaporated and treated with diethyl ether to yield the title compound as crystalline powder.

¹H NMR (DMSO d₆, 400 MHz): 8.73 (s, 1H), 8.23 (d, 1H), 8.18 (s, 1H), 7.82 (d, 2H), 7.65 (m, 1H), 7.58 (dd, 1H), 7.52 (s, 1H), 7.50 (d, 2H), 4.92 (s, 2H), 4.28 (s, 3H), 3.73 (s, 1H), 3.06-2.99 (m, 3H), 2.56 (s, 3H), 1.64 (sext, 2H), 1.30 (d, 6H), 0.94 (t, 3H). HPLC-MS: retention time: 2.55 min (column: Luna), M+1⁺: 516.

### Example 34

### 1-(4-Isopropyl-phenyl)-7-prop-2-ynyloxy-3-(5-trifluoromethyl-1H-benzoimidazol-2-yl)-isoquinoline

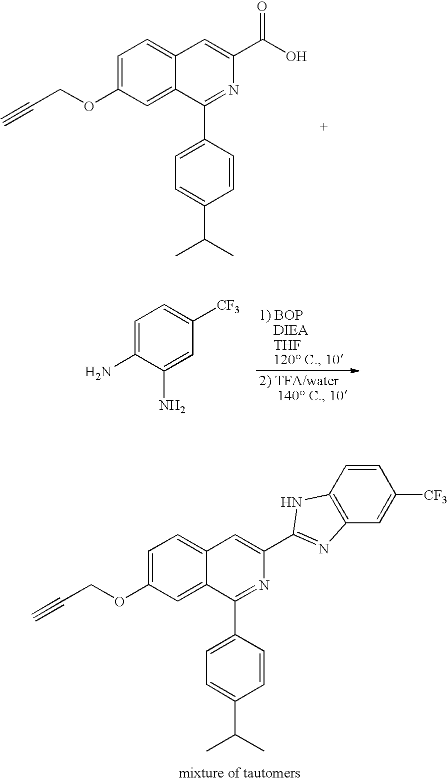

A mixture of 60 mg (0.174 mmol) 1-(4-isopropyl-phenyl)-7-prop-2-ynyloxy-isoquinoline-3-carboxylic acid, 31 mg (0.174 mmol) 4-(trifluoromethyl)-1,2-phenylenediamine, 45 μl (0.26 mmol) N-ethyl-diisopropylamine, and 115 mg (0.26 mmol) BOP in 1 ml THF is heated in a sealed tube by microwave radiation at 120° C. for 10 minutes. After addition of 1 ml water and 1 ml TFA the reaction mixture is heated in a sealed tube at 140° C. for 10 minutes in a microwave oven. After workup with 1 M aqueous NaOH and dichloromethane the crude product is purified by preparative HPLC. Crystalline material is isolated after addition of hexane followed by filtration.

¹H NMR (DMSO d₆, 400 MHz): 13.17 (s, 0.5H), 1.3.15 (s, 0.5H), 8.79 (br, 1H), 8.25 (d, 1H), 8.02 (s, 0.5H), 7.88-7.81 (m, 3H), 7.73 (d, 0.5H), 7.59-7.49 (m, 5H), 4.90 (s, 2H), 3.70 (m, 1H), 3.04 (hept, 1H), 1.31 (d, 6H). HPLC-MS: retention time: 2.65 min (column: Luna), M+1⁺: 486.

The following example is prepared by an analogous procedure:

### Example 35

### 1-(4-Isopropyl-phenyl)-3-(1-methyl-5-trifluoromethyl-1H-benzoimidazol-2-yl)-7-prop-2-ynyloxy-isoquinoline

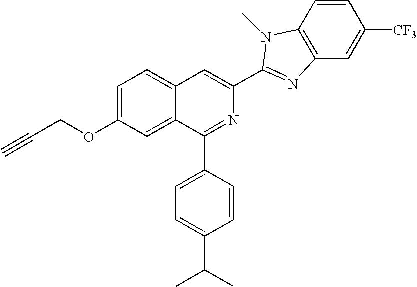

Prepared from N(1)-methyl-4-(trifluoromethyl)-1,2-phenylenediamine.

¹H NMR (DMSO d₆, 400 MHz): 8.78 (s, 1H), 8.25 (d, 1H), 8.06 (s, 1H), 7.86 (d, 1H), 7.82 (d, 2H), 7.66 (d, 1H), 7.62 (dd, 1H), 7.58 (dd, 1H), 7.49 (d, 2H), 4.92 (d, 2H), 4.35 (s, 3H), 3.73 (t, 1H), 3.02 (hept, 1H), 1.30 (d, 6H). HPLC-MS: retention time: 2.62 min (column: Luna), M+1⁺: 500.

### Example 36

### 2-[4-(4-Isopropyl-phenyl)-6-prop-2-ynyloxy-quinazolin-2-yl]-3-methyl-3H-benzoimidazole-5-carboxylic acid ethyl ester

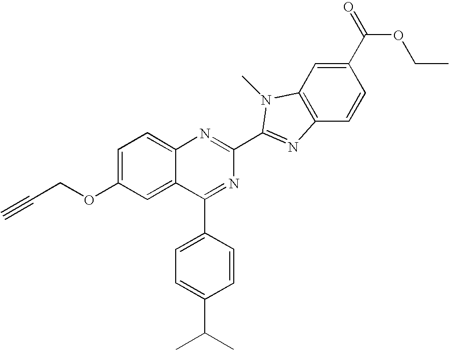

Prepared by method B but heating to 150° C. for 15 min, from 4-(4-isopropyl-phenyl)-6-prop-2-ynyloxy-quinazoline-2-carboxylic acid and 4-amino-3-methylamino-benzoic acid ethyl ester which is obtained from 3-methylamino-4-nitro-benzoic acid by esterification with ethanol/sulfuric acid followed by catalytic hydrogenation (Pd—C (10%) in ethanol) of the nitro group.

¹H NMR (CDCl₃, 400 MHz): 8.27 (d, 1H), 8.24 (m, 1H), 8.03 (dd, 1H), 7.95 (d, 1H), 7.87 (d, 2H), 7.68-7.65 (m, 2H), 7.46 (d, 2H), 4.80 (d, 2H), 4.44 (q, 2H), 4.43 (s, 3H), 3.06 (hept, 1H), 2.62 (t, 1H), 1.45 (t, 3H), 1.36 (d, 6H). HPLC-MS: retention time: 2.35 min (column: Symmetry), M+1⁺: 505.

### Example 37

### 2-[4-(4-isopropyl-phenyl)-6-prop-2-ynyloxy-quinazolin-2-yl]-3-methyl-3H-benzoimidazole-5-carboxylic acid

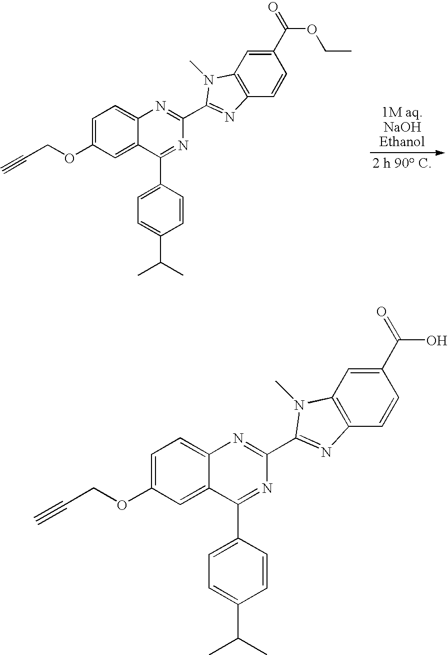

To a solution of 500 mg (0.99 mmol) 2-[4-(4 isopropyl-phenyl)-6-prop-2-ynyloxy-quinazolin-2-yl]-3-methyl-3H-benzoimidazole-5-carboxylic acid ethyl ester in 3 ml ethanol are added 3 ml 1 M aqueous NaOH. After heating to 90° C. (oil bath temperature) for 2 h ethanol is evaporated and the aqueous reaction mixture is acidified by addition of 1 M HCl. The precipitate is filtered off and washed with diethyl ether.

¹H NMR (DMSO d₆, 400 MHz): 8.39 (s, 1H), 8.23 (d, 1H); 7.98 (dd, 1H), 7.95 (d, 2H), 7.86 (d, 1H), 7.85 (dd, 1H), 7.71 (d, 1H), 7.56 (d, 2H), 5.01 (d, 2H), 4.40 (s, 3H), 3.78 (t, 1H), 3.05 (hept, 1H), 1.31 (d, 6H). HPLC-MS: retention time: 2.21 min (column: Symmetry), M+1⁺: 477.

### Example 38

### 2-[4-(4-Isopropyl-phenyl)-6-prop-2-ynyloxy-quinazolin-2-yl]-3-methyl-3H-benzoimidazole-5-carboxylic acid (2-hydroxy-ethyl)-amide

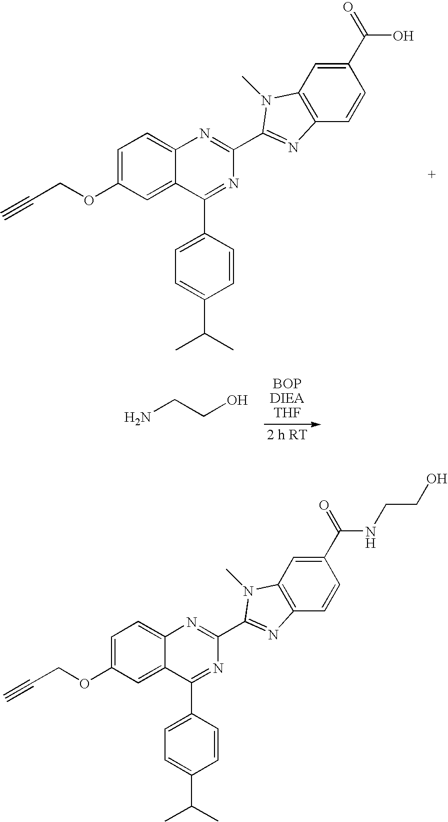

In 1 ml THF 100 mg (0.21 mmol) 2-[4-(4-Isopropyl-phenyl)-6-prop-2-ynyloxy-quinazolin-2-yl]-3-methyl-3H-benzoimidazole-carboxylic acid, 25 μl (0.42 mmol) 2-aminoethanol, 54 μl (0.31 mmol) N-ethyl-diisopropylamine, and 140 mg (0.31 mmol) BOP are dissolved. After 2 h at RT the reaction mixture is subjected to preparative HPLC. The fractions-containing the pure product were extracted against aqueous base with dichloromethane to obtain the title compound.

¹H NMR (DMSO d₆, 400 MHz): 8.57 (t, 1H), 8.33 (s, 1H), 8.22 (d, 1H), 7.93 (d, 2H), 7.90 (dd, 1H), 7.85-7.80 (m, 2H), 7.70 (d, 1H), 7.55 (d, 2H), 5.00 (d, 2H), 4.38 (s, 3H), 3.79 (t, 1H), 3.55 (t, 2H), 3.39 (q, 2H), 3.05 hept, 1H), 1.30 (d, 6H). HPLC-MS: retention time: 2.11 min (column: Symmetry), M+1⁺: 520.

### Example 39

### 2-[4-(4-isopropyl-phenyl)-6-prop-2-ynyloxy-quinazolin-2-yl]-3-methyl-3H-benzoimidazole-5-carboxylic acid dimethylamide

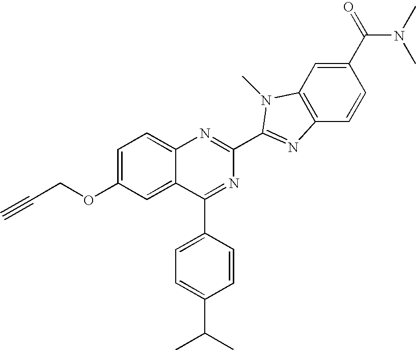

Prepared as described immediately above employing 2 equivalents dimethylamine solution in ethanol.

¹H NMR (DMSO d₆, 400 MHz): 8.19 (d, 1H), 7.92 (d, 2H), 7.83-7.78 (m, 3H), 7.69 (d, 1H), 7.54 (d, 2H), 7.32 (dd, 1H), 4.99 (d, 2H), 4.28 (s, 3H), 3.76 (t, 1H), 3.03 (hept, 1H), 3.00 (s, braod, 6H). 1.30 (d, 6H). HPLC-MS: retention time: 2.17 min (column: Symmetry), M+1⁺: 504.

### Example 40

### 2-[4-(4-Isopropyl-phenyl)-6-prop-2-ynyloxy-quinazolin-2-yl]-3-methyl-3H-benzoimidazole-5-carboxylic acid methylamide

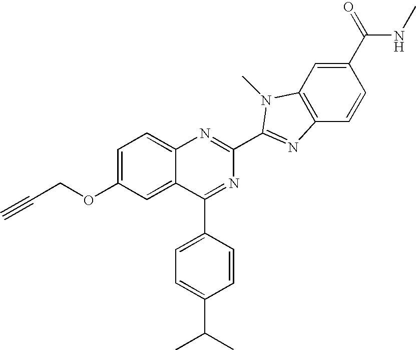

Prepared as described immediately above employing 2 equivalents methylamine solution in ethanol.

¹H NMR (DMSO d₆, 400 MHz): 8.49 (q, 1H), 8.21 (s, 1H), 8.19 (d, 1H), 7.92 (d, 2H), 7.81 (dd, 1H), 7.79 (m, 2H), 7.68 (d, 1H), 7.54 (d, 2H), 4.99 (d, 2H), 4.30 (s, 3H), 3.77 (t, 1H), 3.05 (hept, 1H), 2.84 (d, 3H), 1.30 (d, 6H). HPLC-MS: retention time: 2.16 min (column: Symmetry), M+1⁺: 490.

### Example 41

### Pharmaceutical Formulation

Tablets, comprising as active ingredient 100 mg of one of the active compounds of the preceding examples, respectively, are prepared with the following composition, following standard procedures:

Composition

| | | |
|---|---|---|
| Active ingredient | 100 | mg |
| Crystalline lactose | 240 | mg |
| Avicel | 80 | mg |
| PVPPXL | 20 | mg |
| Aerosil | 2 | mg |
| Magnesium stearate | 5 | mg |
| | 447 | mg |

Manufacture: The active ingredient is admixed with the carrier materials and compressed by means of a tabletting machine (Korsch EKO, piston diameter 10 mm).

Avicel® is microcrystalline cellulose (FMC, Philadelphia, USA).

PVPPXL is polyvinylpolypyrrolidone, cross-linked (BASF, Ludwigshafen, Germany).

Aerosil® is silicium dioxide (Degussa, Germany).

1. A compound of formula (1) or a pharmaceutically acceptable salt thereof or if possible tautomers thereof: 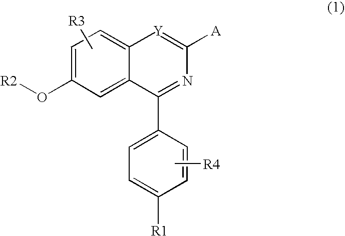
wherein:
Y is CH or N; and
R1 is methyl, ethyl, isopropyl, tert. butyl, or cyclopropyl; and
R2 is methyl, ethyl, propyl, 2-propenyl, or 2-propynyl; and
R3 is hydrogen, chloro, fluoro, bromo, iodo, hydroxy, methoxy, methyl, or trifluoromethyl; and
R4 is hydrogen, chloro, fluoro, bromo, iodo, hydroxy, methoxy, methyl, or trifluoromethyl; and
A is a bi- or tricyclic carbon- or heterocyclic ring system of which one, two, or three cycles can be aryl or heteroaryl and each of which is optionally substituted once or more;
wherein a bicyclic or tricyclic system as described above contains 7 to 13 ring atoms;
wherein a bicyclic or tricyclic system as described above contains 7 to 13 ring atoms one or more of which are selected from the group of O, N, S or C═O;
wherein the optional substituent or substituents are independently selected from the group consisting of phenyl, hydroxyl, trifluoromethyl, methyl, ethyl, branched or unbranched C₃-C₄-alkyl, 2-thiophen, 3-thiophen, hydroxymethyl, hydroxyethyl, methylcarbonyloxymethyl, ethylcarbonyloxyethyl, carboxylmethyl, carboxyethyl, methoxycarbonyl, ethoxycarbonyl, branched or unbranched C₃-C₄-alkyloxycarbonyl, methoxy, ethoxy, branched or unbranched C₃-C₄-alkyloxy, branched or unbranched C₃-C₅-alkyloxycarbonylmethyl, branched or unbranched C₃-C₅-alkyloxycarbonylethyl, methylcarbonyl, ethylcarbonyl, branched or unbranched C₃-C₄-alkylcarbonyl, phenylcarbonyl, chloro, fluoro, bromo, iodo, cyano, nitro, furanyl, pyrrolyl, thiazolyl, benzothiazolyl, pyridinyl, amino, sulfanyl, sulfonyl, oxycarbonyl, sulfinyl, aminosulfonyl, sulfonylamino, carbonyl, carbonyloxy, carbonyl amino, carboxyl, acyl, acylamino, or carbamoyl;
wherein the optional substituents or substituents as defined above can be again optionally substituted by one or several substituents of the group consisting of methyl, ethyl, branched or unbranched C₃-C₄ alkyl, fluoro, chloro, bromo, iodo, trifluoromethyl, hydroxy, methoxy, amino, alkylamino, dialkylamino, cyano, carboxyl, methylcarboxyl, ethylcarboxyl, branched or unbranched C₃-C₅alkylcarboxyl, acetyl, 2-hydroxyethyl and 3-hydroxypropyl.

2. A compound of formula (3) or a pharmaceutically acceptable salt thereof: 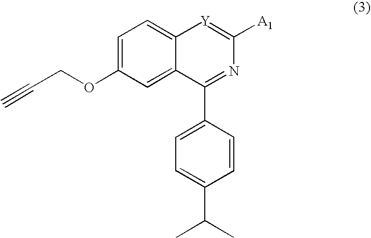
wherein:
Y is CH or N; and
A₁ is being independently selected from the group consisting of 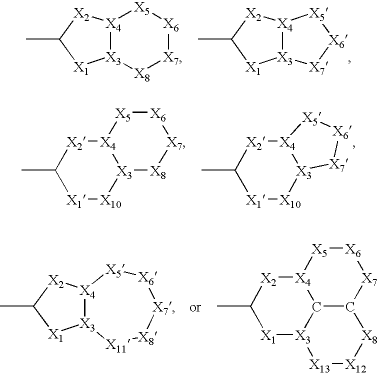
wherein
X₁, X₂ is each independently N, O, S, or C; and
X₁′, X₂′ is each independently C, N, or O; and
X₃, X₄ is each independently C or N; and
X₅, X₆, X₇, X₈, X₁₂, X₁₃ is each independently C, N, O, or C═O; and
X₅′, X₆′, X₇′, X₁₁′ is each independently C, N, O, S, C═O; and
X₁₀ is C═O, C, or N; and
wherein the bonds within the cycles are single, double or aromatic; and
wherein the cyclic groups can be optionally substituted as follows: 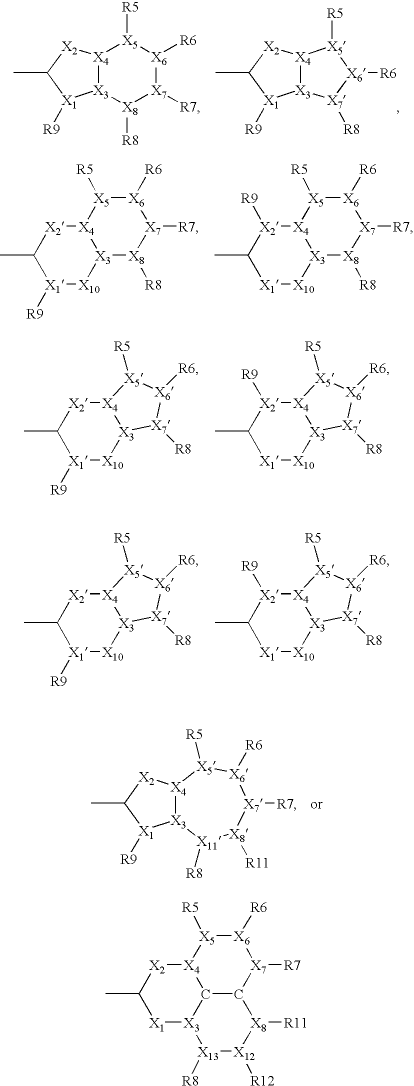
wherein R5, R6, R7, R8, R9, R11, or R12 is each independently selected from the group consisting of phenyl, hydroxyl, trifluoromethyl, methyl, ethyl, branched or unbranched C₃-C₄-alkyl, 2-thiophen, 3-thiophen, hydroxymethyl, hydroxyethyl, methylcarbonyloxymethyl, ethylcarbonyloxyethyl, carboxymethyl, carboxyethyl, methoxy, ethoxy, branched or unbranched C₃-C₄-alkyloxy, branched or unbranched C₃-C₄-alkyloxycarbonylmethyl, branched or unbranched C₃-C₄-alkyloxycarbonylethyl, methylcarbonyl, ethylcarbonyl, branched or unbranched C₃-C₄-alkylcarbonyl, phenylcarbonyl, chloro, fluoro, bromo, iodo, cyano, nitro, furanyl, thienyl, pyrrolyl, thiazolyl, benzothiazolyl, pyridinyl, amino, sulfanyl, sulfonyl, amino, oxycarbonyl, hydroxyl, sulfinyl, aminosulfonyl, sulfonylamino, carbonyl, carbonyloxy, carbonyl amino, carboxyl, acyl, acylamino, and carbamoyl; or from ethoxycarbonyl, N-(2-hydroxyethyl)-aminocarbonyl, N-(3-hydroxypropyl)-aminocarbonyl, N-methylaminocarbonyl and N,N-dimethylaminocarbonyl; and
wherein neighbored residues of R5, R6, R7, R8, R9, R11, or R12 can form together an alicycle, heterocycle or an aromatic or heteroaromatic cycle which can itself be optionally substituted by methyl, ethyl, branched or unbranched C₃-C₄-alkyl; wherein said aromatic cycle can then form together with the other bicycle e.g. perimidinyl, e.g. 2-perimidinyl or said heteroaromatic cycle can e.g. be phenanthrolinyl, naphtothienyle such as naphtho[2,3-b]thienyl or naphtooxazole such as naphtho[1,2-d]oxazole.

3. A compound or pharmaceutically acceptable salt thereof selected from the group consisting of 1-{2-[4-(4-Isopropyl-phenyl)-prop-2-ynyloxy-quinazolin-2-yl]-1,6-dimethyl-1H-benzoimidazol-5-yl}butan-1-one; 4-(4-Isopropyl-phenyl)-2-(4-methyl-1H-benzoimidazol-2-yl)-6-prop2-ynyloxy-quinazoline; 2-[4-(4-Isopropyl-phenyl)-6-prop-2-ynyloxy-quinazolin-2-yl]-3H-benzoimidazole-5-carbonitrile; 2-(1-Isobutyl-1H-benzoimidazol-2-yl)-4-(4-isopropyl-phenyl)-6-prop-2-ynyloxy-quinazoline; 2-(5-Fluoro-1-methyl-1H-benzoimidazol-2-yl)-4-(4-isopropyl-phenyl)-6-prop-2-ynyloxy-quinazoline; 4-(4-Isopropyl-phenyl)-2-(5-methoxy-1-methyl-1H-benzoimidazol-2-yl)-6-prop-2-ynyloxy-quinazoline; 2-(4,6-Dimethyl-1H-benzoimidazol-2-yl)-4-(4-isopropyl-phenyl)-6-prop-2-ynyloxy-quinazoline; 4-(4-Isopropyl-phenyl)-2-(1-methyl-1H-benzoimidazol-2-yl)-6-prop-2-ynyloxy-quinazoline; 4-(4-Isopropyl-phenyl)-2-(1-methyl-1H-imidazo[4,5-b]pyridin-2-yl)-6-prop-2-ynyloxy-quinazoline; 2-(1H-Imidazo[4,5-b]pyridin-2-yl)-4-(4-isopropyl-phenyl)-6-prop-2-ynyloxy-quinazoline; 4-(4-isopropyl-phenyl)-6-prop-2-ynyloxy-2-(9H-purin-8-yl)-quinazoline; 4-(4-Isopropyl-phenyl)-2-(1-phenyl-1H-benzoimidazol-2-yl)-6-prop-2-ynyloxy-quinazoline; 1-{1-Butyl-2-[4-(4-isopropyl-phenyl)-6-prop-2-ynyloxy-quinazolin-2-yl]-6-methyl-1H-benzoimidazol-5-yl}-butan-1-one; 4-(4-Isopropylphenyl)-6-prop-2-ynyloxy-2-(6-trifluoromethyl-1H-benzoimidazol-2-yl)-quinazoline; 4-(4-Isopropylphenyl)-2-(6-methoxy-1H-benzoimidazol-2-yl)-6-prop-2-ynyloxy-quinazoline; 2-(6-Chloro-1H-benzoimidazol-2-yl)-4-(4-isopropyl-phenyl)-6-prop-2-ynyloxy-quinazoline; 2-[4-(4-Isopropyl-phenyl)-6-prop-2-ynyloxy-quinazolin-2-yl]-3H-benzoimidazole-5-carboxylic acid ethyl ester; {2-[4-(4-Isopropyl-phenyl)-6-prop-2-ynyloxy-quinazolin-2-yl]-3H-benzoimidazol-5-yl}-phenyl-methanone; 2-(6-Bromo-1H-benzoimidazol-2-yl)-4-(4-isopropyl-phenyl)-6-prop-2-ynyloxy-quinazoline; 2-(6-Fluoro-1H-benzoimidazol-2-yl)-4-(4-isopropyl-phenyl)-6-prop-2-ynyloxy-quinazoline; 2-(6-Chloro-5-fluoro-1H-benzoimidazol-2-yl)-4-(4-isopropyl-phenyl)-6-prop-2-ynyloxy-quinazoline; 4-(4-Isopropylphenyl)-2-(1-methyl-5-trifluoromethyl-1H-benzoimidazol-2-yl)-6-prop-2-ynyloxy-quinazoline; 2-[4-(4-Isopropyl-phenyl)-6-prop-2-ynyloxy-quinazolin-2-yl]-1-methyl-1H-benzoimidazole-5-carboxylic acid ethyl ester; 1-{2-[4-(4-Isopropyl-phenyl)-6-prop-2-ynyloxy-quinazolin-2-yl]-1-methyl-1H-benzoimidazol-5-yl}-ethanone; 2-(5,6-Difluoro-1H-benzoimidazol-2-yl)-4-(4-isopropyl-phenyl)-6-prop-2-ynyloxy-quinazoline; 1-{2-[4-(4-Isopropyl-phenyl)-6-prop-2-ynyloxy-quinazolin-2-yl]-3H-benzoimidazol-4-yl}-ethanone; 2-[4-(4-Isopropyl-phenyl)-6-prop-2-ynyloxy-quinazolin-2-yl]-1H-perimidine; 4′-(4-Isopropyl-phenyl)-6′-prop-2-ynyloxy-3H-[2,2′]biquinazolinyl-4-one; 4-(4-Isopropylphenyl)-6-prop-2-ynyloxy-2-(5-trifluoromethyl-benzothiazol-2-yl)-quinazoline; 2-Benzothiazol-2-yl-4-(4-isopropyl-phenyl)-6-prop-2-ynyloxy-quinazoline; 2-(6-Chloro-benzooxazol-2-yl)-4-(4-isopropyl-phenyl)-6-prop-2-ynyloxy-quinazoline; 2-[4-(4-Isopropyl-phenyl)-6-prop-2-ynyloxy-quinazolin-2-yl]-naphtho[1,2-d]oxazole; 1-{2-[1-(4-Isopropyl-phenyl)-7-prop-2-ynyloxy-isoquinolin-3-yl]-1,6-dimethyl-1H-benzoimidazol-5-yl}-butan-1-one; 1-(4-Isopropyl-phenyl)-7-prop-2-ynyloxy-3-(5-trifluoromethyl-1H-benzoimidazol-2-yl)-isoquinoline; 1-(4-Isopropyl-phenyl)-3-(1-methyl-5-trifluoromethyl-1H-benzoimidazol-2-yl)-7-prop-2-ynyloxy-isoquinoline, 5-ethoxycarbonyl-3-methyl-3H-benzoimidazole-2-yl, 5-carboxyl-3-methyl-3H-benzoimidazole-2-yl, 5-[N-(2-hydroxyethyl)aminocarbonyl]-3-methyl-3H-benzoimidazol-2-yl, 5-(N,N-dimethyl-carbamoyl)-3-methyl-3H-benzimidazol-2-yl and 5-(N-methyl-carbamoyl)-3-methyl-3H-benzimidazol-2-yl.

4. A compound selected from the group consisting of 1-{2-[4-(4-Isopropyl-phenyl)-6-prop-2-ynyloxy-quinazolin-2-yl]-1,6-dimethyl-1H-benzoimidazol-5-yl}-butan-1-one; 4-(4-Isopropyl-phenyl)-2-(4-methyl-1H-benzoimidazol-2-yl)-6-prop2-ynyloxy-quinazoline; 2-[4-(4-Isopropyl-phenyl)-6-prop-2-ynyloxy-quinazolin-2-yl]-3H-benzoimidazole-5-carbonitrile; 2-(1-Isobutyl-1H-benzoimidazol-2-yl)-4-(4-isopropyl-phenyl)-6-prop-2-ynyloxy-quinazoline; 2-(5-Fluoro-1-methyl-1H-benzoimidazol-2-yl)-4-(4-isopropyl-phenyl)-6-prop-2-ynyloxy-quinazoline; 4-(4-Isopropyl-phenyl)-2-(5-methoxy-1-methyl-1H-benzoimidazol-2-yl)-6-prop-2-ynyloxy-quinazoline; 2-(4,6-Dimethyl-1H-benzoimidazol-2-yl)-4-(4-isopropyl-phenyl)-6-prop-2-ynyloxy-quinazoline; 4-(4-Isopropyl-phenyl)-2-(1-methyl-1H-benzoimidazol-2-yl)-6-prop-2-ynyloxy-quinazoline; 4-(4-Isopropyl-phenyl)-2-(1-methyl-1H-imidazo[4,5-b]pyridin-2-yl)-6-prop-2-ynyloxy-quinazoline; 2-(1H-Imidazo[4,5-b]pyridin-2-yl)-4-(4-isopropyl-phenyl)-6-prop-2-ynyloxy-quinazoline; 4-(4-Isopropyl-phenyl)-6-prop-2-ynyloxy-2-(9H-purin-8-yl)-quinazoline; 4-(4-Isopropyl-phenyl)-2-(1-phenyl-1H-benzoimidazol-2-yl)-6-prop-2-ynyloxy-quinazoline; 1-{1-Butyl-2-[4-(isopropyl-phenyl)-6-prop-2-ynyloxy-quinazolin-2-yl]-6-methyl-1H-benzoimidazol-5-yl}-butan-1-one; 4-(4-Isopropyl-phenyl)-6-prop-2-ynyloxy-2-(6-trifluoromethyl-1H-benzoimidazol-2-yl)-quinazoline; 4-(4-Isopropyl-phenyl)-2-(6-methoxy-1H-benzoimidazol-2-yl)-6-prop-2-ynyloxy-quinazoline; 2-(6-Chloro-1H-benzoimidazol-2-yl)-4-(4-isopropyl-phenyl)-6-prop-2-ynyloxy-quinazoline; 2-[4-(4-Isopropyl-phenyl)-6-prop-2-ynyloxy-quinazolin-2-yl]-3H-benzoimidazole-5-carboxylic acid ethyl ester; {2-[4-(4-Isopropyl-phenyl)-prop-2-ynyloxy-quinazolin-2-yl]-3H-benzoimidazol-5-yl}-phenyl-methanone; 2-(6-Bromo-1H-benzoimidazol-2-yl)-4-(4-isopropyl-phenyl)-6-prop-2-ynyloxy-quinazoline; 2-(6-Fluoro-1H-benzoimidazol-2-yl)-4-(4-isopropyl-phenyl)-6-prop-2-ynyloxy-quinazoline; 2-(6-Chloro-5-fluoro-1H-benzoimidazol-2-yl)-4-(4-isopropyl-phenyl)-6-prop-2-ynyloxy-quinazoline; 4-(4-Isopropyl-phenyl)-2-(1-methyl-5-trifluoromethyl-1H-benzoimidazol-2-yl)-6-prop-2-ynyloxy-quinazoline; 2-[4-(4-Isopropylphenyl)-6-prop-2-ynyloxy-quinazolin-2-yl]-1-methyl-1H-benzoimidazole-5-carboxylic acid ethyl ester; 1-{2-[4-(4-Isopropyl-phenyl)-6-prop-2-ynyloxy-quinazolin-2-yl]-1-methyl-1H-benzoimidazol-5-yl}-ethanone; 2-(5,6-Difluoro-1H-benzoimidazol-2-yl)-4-(4-isopropyl-phenyl)-6-prop-2-ynyloxy-quinazoline; 1-{2-[4-(4-Isopropyl-phenyl)-6-prop-2-ynyloxy-quinazolin-2-yl]-3H-benzoimidazol-4-yl}-ethanone; 2-[4-(4-Isopropyl-phenyl)-6-prop-2-ynyloxy-quinazolin-2-yl]-1H-perimidine; 4′-(4-Isopropyl-phenyl)-6′-prop-2-ynyloxy-3H-[2,2′]biquinazolinyl-4-one; 4-(4-Isopropyl-phenyl)-6-prop-2-ynyloxy-2-(5-trifluoromethyl-benzothiazol-2-yl)-quinazoline; 2-Benzothiazol-2-yl-4-(4-isopropyl-phenyl)-6-prop-2-ynyloxy-quinazoline; 2-(6-Chloro-benzooxazol-2-yl)-4-(4-isopropyl-phenyl)-6-prop-2-ynyloxy-quinazoline;
or a pharmaceutically acceptable salt thereof.

5. A compound selected from the group consisting of 2-[4-(4-Isopropyl-phenyl)-8-prop-2-ynyloxy-quinazolin-2-yl]-3-methyl-3H-benzoimidazole-5-carboxylic acid ethyl ester, 2-[4-(4-Isopropyl-phenyl)-6-prop-2-ynyloxy-quinazolin-2-yl]-3-methyl-3H-benzoimidazole-5-carboxylic acid, 2-[4-(4-Isopropyl-phenyl)-6-prop-2-ynyloxy-quinazolin-2-yl]-3-methyl-3H-benzoimidazole-5-carboxylic acid (2-hydroxy-ethyl)-amide, 2-[4-(4-Isopropyl-phenyl)-6-prop-2-ynyloxy-quinazolin-2-yl]-3-methyl-3H-benzoimidazole-5-carboxylic acid dimethylamide and 2-[4-(4-Isopropyl-phenyl)-6-prop-2-ynyloxy-quinazolin-2-yl]-3-methyl-3H-benzoimidazole-5-carboxylic acid methylamide;
or a pharmaceutically acceptable salt thereof.

6. A pharmaceutical composition for preventing or treating bone conditions comprising a compound of claim 1 in association with a pharmaceutically acceptable excipient, diluent or carrier.

7. (canceled)

8. A process for preparation of a compound of claim 2 in free or salt form, comprising the step of:
reacting a compound of formula (10) 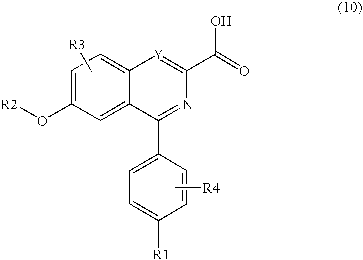
with a compound selected from the group consisting of 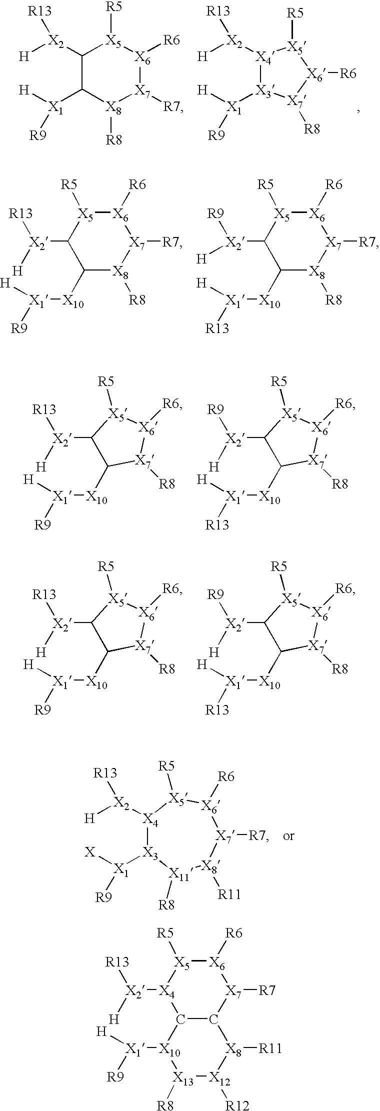
wherein the residues R1 to R12, Y are as defined in claim 2; and
wherein the H forms part of an amino, thiol, hydroxy, or basic CH, group, the reaction being carried out in the presence of a coupling reagent; and
wherein R13 is an electron lone pair or H, in case R9 is not H, R14 must be H.

9. A combination comprising a therapeutically effective amount of a compound of claim 1 and a second drug substance selected from the group consisting of: calcium, a calcitonin or an analogue or derivative thereof, a steroid hormone, a partial estrogen agonist or estrogen-gestagen combination, a SERM (Selective Estrogen Receptor Modulator), a RANKL antibody, a cathepsin K inhibitor, vitamin D or an analogue thereof or PTH, a PTH fragment or a PTH derivative for simultaneous, separate or sequential treatment.

10. A method for preventing or treating bone conditions which are associated with increased calcium depletion or resorption or in which stimulation of bone formation and calcium fixation in the bone is desirable in which an effective amount of a compound of compound of claim 1 is administered to a patient in need of such treatment.

11. (canceled)

12. A pharmaceutical composition for preventing or treating bone conditions comprising a compound of claim 2 in association with a pharmaceutically acceptable excipient, diluent or carrier.

13. A combination comprising a therapeutically effective amount of a compound of claim and a second drug substance selected from the group consisting of: calcium, a calcitonin or an analogue or derivative thereof, a steroid hormone, a partial estrogen agonist or estrogen-gestagen combination, a SERM (Selective Estrogen Receptor Modulator), a RANKL antibody, a cathepsin K inhibitor, vitamin D or an analogue thereof or PTH, a PTH fragment or a PTH derivative for simultaneous, separate or sequential treatment.

14. A method for preventing or treating bone conditions which are associated with increased calcium depletion or resorption or in which stimulation of bone formation and calcium fixation in the bone is desirable in which an effective amount of a compound of compound of claim 2 is administered to a patient in need of such treatment.
